(19) United States
(12) Patent Application Publication
(10) Pub. No.: US 2023/0032020 A1
(43) Pub. Date: Feb. 2, 2023

(54) SMALL-MOLECULE COVALENT INHIBITION OF RAL GTPASES

(71) Applicant: THE TRUSTEES OF INDIANA UNIVERSITY, Bloomington, IN (US)

(72) Inventors: Samy MEROUEH, Carmel, IN (US); Khuchtumur BUM-ERDENE, Indianapolis, IN (US); Giovanni GONZALEZ-GUTIERREZ, Bloomington, IN (US)

(21) Appl. No.: 17/782,870

(22) PCT Filed: Dec. 18, 2020
(86) PCT No.: PCT/US2020/066082

§ 371 Date: Jun. 6, 2022

Related U.S. Application Data
(60) Provisional application No. 62/950,259, filed on Dec. 19, 2019.

(51) Int. Cl. A61K 31/357; A61K 31/4184; A61K 31/4433; A61K 31/4412

(52) U.S. Cl. CPC A61K 31/357; A61K 31/4184; A61K 31/4433; A61K 31/4412

(57) ABSTRACT

Disclosed herein are Ral-antagonist compounds that covalently bind to binding sites in RalA, and efficaciously inhibit Ral activity. The compounds include aryl sulfonyl fluoride compounds of the general structure of wherein X and Y are independently C or N, and R₄ is C₁-C₄ alkyl, —OCH₃, —OCH₂CH₃, —OCH(CH₃)₂, —(SO₂)CH₃, —OH, or halo. These compounds expand Ral-inhibiting therapeutic options for treating Ral-driven cancers and one embodiment of the present disclosure is directed to the use of such compounds to treat cancer.

### CROSS REFERENCE TO RELATED APPLICATIONS

This application claims priority to U.S. Provisional Patent Application No. 62/950,259 filed on Dec. 19, 2019, the disclosure of which is expressly incorporated herein.

### STATEMENT REGARDING FEDERALLY SPONSORED RESEARCH AND DEVELOPMENT

This invention was made with government support under CA197928 awarded by the National Institutes of Health. The government has certain rights in the invention.

### BACKGROUND OF THE DISCLOSURE

Ras and Ral GTPases are active in cellular signal transduction in pathways that control cell growth and division. Ras and Ral GTPases cycle between an active GTP-bound complex that triggers such cellular signaling and an inactive GDP-bound complex that does not trigger such signaling. Mutations in ras or ral genes can result in permanently activated Ras or Ral proteins, which cause overactive signaling for cell growth and division. In this manner, mutant Ras and Ral proteins contribute to the development of many types of human cancers, and are particularly common in certain types of cancer such as pancreatic cancer and lung cancer. Moreover, Ras and Ral GTPases have been among the most intractable targets in cancer drug discovery. A significant problem is that Ras and Ral GTPases do not possess a native druggable binding pocket that can be used as a target for the development of drugs that inhibit mutant Ras and/or Ral activity. Thus, compounds that effectively inhibit mutant Ras and/or Ral activity may have broad medical and societal benefits as therapeutic agents for the treatment of Ras- and/or Ral-driven cancers.

It also would be desirable to develop compounds that specifically inhibit mutant Ral activity, since substantial evidence indicates a supporting a role for Ral GTPases in cancer that is both dependent and independent of Ras. Further, it would be desirable to develop compounds that effectively inhibit mutant Ral activity in substantial proportion of Ral-driven tumors. Benefits of this approach may include efficacious cancer treatment for a greater number of patients than currently is possible with known compounds that are intended to target mutant Ral or Ral activity, thus improving clinical outcomes.

### SUMMARY

One embodiment of the present disclosure is directed to Ral-antagonist compounds that covalently bind to new well-defined druggable binding sites in Ral GTPase RalA, and efficaciously inhibit Ral activity by inhibiting a Ral guanine exchange factor. The new druggable binding sites disclosed herein advantageously are present in Ral in a large proportion of Ral-driven cancers, thereby expanding a Ral-inhibiting treatment option to a greater number of cancer patients than is currently possible with known inhibitors of GTPases of the Ras superfamily The present disclosure is further directed to novel methods of identifying such Ral-antagonist compounds and to methods of treating patients with Ral-driven cancers.

In accordance with one embodiment of the present disclosure, aryl sulfonyl fluoride compounds have been identified that form covalent bonds with Tyr-82 of RalA and thus inhibit Ral guanine exchange factor 2 (Rgl2)-mediated Ral nucleotide exchange. Advantageously, such covalent inhibitors do not require deep hydrophobic pockets to engage a target as long as the reactive group of these compounds can rapidly form a covalent bond with an amino acid side chain. In addition, Tyr-82 is a non-mutant RalA residue that is present in most mutant and wild-type RalA proteins, absent a mutation of Tyr-82 itself. Therefore, regardless of the specific oncogenic mutation of any given RalA, Tyr-82 is likely to be present, which makes Tyr-82 a widespread target for Ral inhibition.

In accordance with one embodiment a method of identifying a small-molecule compounds capable of covalent bonding with an amino acid residue of a Ral GTPase to inhibit Ral GTPase activity is provided. In one embodiment the method comprises screening compounds having a core structure of:

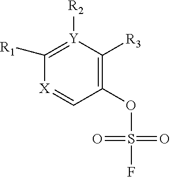

wherein X and Y are independently C or N, R₁ is an five or six membered ring selected from an optionally substituted heterocylic, optionally substituted aryl or optionally substituted heteroaryl, R₂ is H, C₁-C₄ alkyl, or CF₃; and R₃ is H, or R₂ and R₃ together with the atom to which they are attached form a 6 membered heterocyclic or aryl ring, optionally a 1,4 dioxane, hexacyclic, or morpholino ring to identify compounds that covalent bond with an amino acid residue, optionally Tyr-82, of the Ral GTPase. In one embodiment the method comprises:

incubating the small-molecule compound with a sample of the Ral GTPase;

conducting one or more assays comprising at least one of:

i) a fluorescence-based guanine nucleotide exchange assay indicative of inhibition of Rgl2-mediated exchange of Ral-bound GDP;

ii) a time-dependent assay measuring inhibition of Rgl2-mediated exchange of Ral-bound GDP;

iii) a protein dialysis assay measuring inhibition of Rgl2-mediated exchange of Ral-bound GDP; and

iv) intact protein mass spectrometry showing that the small-molecule compound forms a covalent bond with the Ral GTPase at the amino acid residue; and

identifying the small-molecule compound as covalent bonding with the amino acid residue of the Ral GTPase based on the outcomes of the one or more of assays i)-iv).

In accordance with one embodiment an aryl sulfonyl fluoride suitable for use in accordance with the present disclosure is provided having the structure of Formula I:

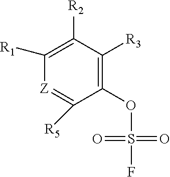

wherein

R₁ is H,

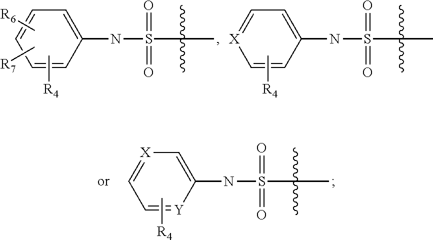

R₂ is H, C₁-C₄ alkyl, or CF₃,

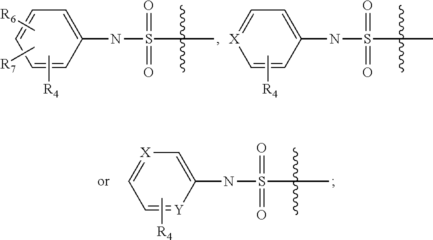

with the proviso that only one of R₁ or R₂ is

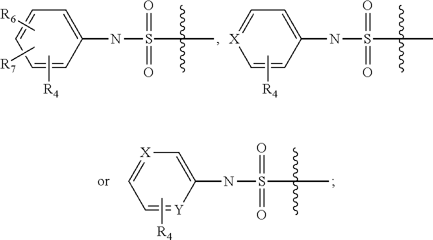

R₃ is H, C₁-C₄ alkyl, —OCH₃, —OCH₂CH₃, —OCH(CH₃)₂, or R₂ and R₃ together with the atom to which they are attached form a 6 membered heterocyclic, or a 6 membered aryl ring, optionally a 1,4 dioxane or hexacyclic ring;

R₄ is C₁-C₄ alkyl, —OCH₃, —OCH₂CH₃, —OCH(CH₃)₂, —(SO₂)CH₃, —OH, or halo;

R₅ is H or ═O;

R₆ and R₇ are independently halo;

X, Y and Z are independently C or N, optionally wherein Z is N and X and Y are each C, optionally wherein X is N and Y and Z are each C and optionally wherein X, Y and Z are each C.

In accordance with one embodiment an aryl sulfonyl fluoride suitable for use in accordance with the present disclosure is provided having the structure of

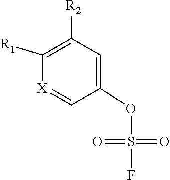

wherein

R₁ and R₂ are independently H or

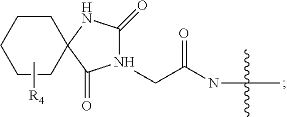

with the proviso that one of R₁ or R₂ is H;

R₄ is C₁-C₄ alkyl, —OCH₃, —OCH₂CH₃, —OCH(CH₃)₂, —(SO₂)CH₃, —OH, or halo, optionally wherein R₄ is C₁-C₂ alkyl or —OCH₃; and

X is C or N.

In one embodiment an aryl sulfonyl fluoride suitable for use in accordance with the present disclosure is provided having the structure of:

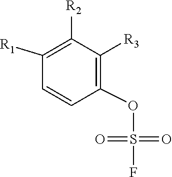

wherein

R₁ is

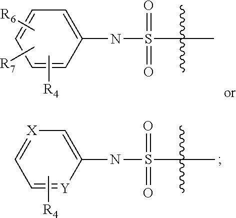

R₃ is H, C₁-C₄ alkyl, —OCH₃, —OCH₂CH₃, —OCH(CH₃)₂;

R₂ is H, or R₂ and R₃ together with the atom to which they are attached form a 6 membered aryl ring, a 1,4 dioxane or hexacyclic ring;

R₄ is C₁-C₄ alkyl, —OCH₃, —OCH₂CH₃, —OCH(CH₃)₂, —(SO₂)CH₃, —OH, or halo;

R₆ and R₇ are independently halo or H, optionally wherein said halo is Cl or F;

X and Y are independently C or N, optionally wherein X is N and Y is C.

In one embodiment an aryl sulfonyl fluoride suitable for use in accordance with the present disclosure is provided having the structure of:

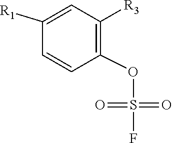

wherein

R₁ is

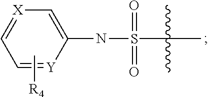

R₃ is H, C₁-C₄ alkyl, —OCH₃, —OCH₂CH₃, or —OCH(CH₃)₂;

R₄ is —OCH₃, —OCH₂CH₃, —OCH(CH₃)₂, —OH, or halo;

X and Y are independently C or N, optionally wherein X is N and Y is C.

In one embodiment an aryl sulfonyl fluoride suitable for use in accordance with the present disclosure is provided having the structure of:

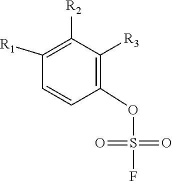

wherein

R₁ is

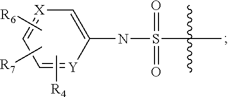

R₂ and R₃ together with the atom to which they are attached form a 1,4 dioxane or hexacyclic ring;

R₄ is —OCH₃, —OCH₂CH₃, —OCH(CH₃)₂, or —OH;

R₆ and R₇ are independently H or halo, optionally wherein said halo is F or Cl, and

X and Y are independently C or N, optionally wherein X is N and Y is C. In a further embodiment R₆ is H, X is N and Y is C.

In one embodiment the aryl sulfonyl fluoride is compound having the structure of:

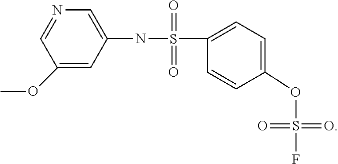 [SMILES: COc1cncc(NS(=O)(=O)c2ccc(OS(=O)(=O)F)cc2)c1]

In one embodiment the aryl sulfonyl fluoride is compound having the structure of any one of compounds 1-23 of Table 1. In one embodiment the aryl sulfonyl fluoride is compound having the structure of any one of compounds SOF564, SOF365, SOF366, SOF367, SOF368, SOF369, SOF370, SOF371, SOF376, SOF377, SOF378, SOF379, SOF380, SOF381, SOF382, SOF531, SOF532, SOF533, SOF534, SOF535 and SOF536.

Additional embodiments, features, and advantages of the disclosure will be apparent from the following detailed description and through practice of the disclosure. The compounds and methods of the present disclosure can be described as embodiments in any of the enumerated clauses set forth herein. It will be understood that any of the embodiments described herein can be used in connection with any other embodiments described herein to the extent that the embodiments do not contradict one another.

### BRIEF DESCRIPTION OF THE DRAWINGS

FIGS. 1A-1F collectively illustrate the effectiveness of an aryl sulfonyl fluoride (Compound 1) to inhibit Ral guanine exchange factor 2 (Rgl2)-mediated Ral nucleotide exchange. FIG. 1A provides the structure of one aryl sulfonyl fluoride suitable for use in the present invention and the reaction mechanism resulting in the covalent linkage of the aryl sulfonyl fluoride to Tyr-82 of RalA. FIG. 1B is a graph presenting data that illustrates inhibition of Rgl2-mediated guanine nucleotide exchange of RalB by 100 μM after 24 h incubation with the compound of FIG. 1A at 4° C. FIG. 1C is a graph presenting data that illustrates inhibition of Rgl2-mediated guanine nucleotide exchange of RalB Tyr82Phe mutant by 100 μM after 24 h incubation with the compound of FIG. 1A at 4° C. FIG. 1D is a graph illustrating the concentration-dependent percent inhibition of Rgl2-mediated guanine nucleotide exchange of RalB after 24 h incubation with the compound of FIG. 1A at 4° C. FIG. 1E is a graph illustrating the concentration-dependent percent inhibition of Rgl2-mediated guanine nucleotide exchange of RalB after 0.5, 6, 24 and 48 h incubation with the compound of FIG. 1A at 4° C. FIG. 1F is a graph illustrating the percent inhibition of Rgl2-mediated guanine nucleotide exchange of RalB after 24 h incubation with the compound of FIG. 1A at 4° C. followed by 24 h dialysis against assay buffer at 4° C. relative to the percent inhibition in the absence of the dialysis step.

FIGS. 2A & 2B: FIG. 2A is a two-dimensional ligand interaction map of covalently bound Compound 1 (as shown in FIG. 1A and Table 1) in the druggable pocket at the switch II loop of RalA generated using Maestro. FIG. 2B illustrates the percent inhibition of Rgl2-mediated guanine nucleotide exchange of RalB, RalB Tyr82Phe mutant, RalB Ser85Ala mutant, Thr69Ala mutant and RalA by 100 μM of compound 1 after 24 h incubation at 4° C.

FIG. 3 is a bar graph illustrating the percent inhibition of Rgl2-mediated guanine nucleotide exchange of RalB, RalB Tyr82Phe mutant and RalA and Sos-mediated guanine nucleotide exchange H-Ras and K-Ras by 50 uM compounds after 24 h incubation at 4° C. The structure of compounds 2-23 is provided in Table 1.

FIGS. 4A-4K: FIG. 4A illustrates percent inhibition of Rgl2-mediated guanine nucleotide exchange of RalB by 100 μM of the indicated compounds after 24 h incubation at 4° C. followed by 24 h dialysis against assay buffer at 4° C. FIGS. 6B-6K illustrate concentration-dependent percent inhibition of Rgl2-mediated guanine nucleotide exchange of RalB after 0.5, 6, 24 and 48 h incubation at 4° C. with compounds 2, 4, 5, 6, 7, 11, 15, 21, 22 and 23, respectively. The structure of each of compounds 2, 4, 5, 6, 7, 11, 15, 21, 22 and 23 is provided in Table 1.

FIG. 5 illustrates the structure and the concentration-dependent percent inhibition of Rgl2-mediated guanine nucleotide exchange of RalB and RalB Tyr82Phe mutant after 24 h incubation at 4° C. with SOF 364.

FIG. 6 illustrates the structure and the concentration-dependent percent inhibition of Rgl2-mediated guanine nucleotide exchange of RalB and RalB Tyr82Phe mutant after 24 h incubation at 4° C. with SOF 365.

FIG. 7 illustrates the structure and the concentration-dependent percent inhibition of Rgl2-mediated guanine nucleotide exchange of RalB and RalB Tyr82Phe mutant after 24 h incubation at 4° C. with SOF 366.

FIG. 8 illustrates the structure and the concentration-dependent percent inhibition of Rgl2-mediated guanine nucleotide exchange of RalB and RalB Tyr82Phe mutant after 24 h incubation at 4° C. with SOF 367. SOF-367 shows higher stability than Compound 1, and inhibits RalA and RalB exchange.

FIG. 9 illustrates the concentration-dependent inhibition by SOF-367 and SOF-531 Pa03C of pancreatic cancer cell growth in 3-D co-culture with fibroblasts, relative to treatment with media or DMSO.

FIG. 10 is a bar graph demonstrating the concentration-dependent effectiveness of SOF-367 to inhibit Mia-Paca2 cancer cell invasion (at concentration of 1, 10 and 50 uM) relative to SOF-344 at 50 uM and control.

FIG. 11 is a bar graph demonstrating the concentration-dependent effectiveness of SOF-367 to inhibit Aspc-1 cancer cell invasion (at concentration of 1, 10 and 50 uM) relative to SOF-344 at 50 uM and control.

FIGS. 12A & 12B are bar graphs demonstrating SOF-367 is not cytotoxic to Mia-Paca2 cancer cells (FIG. 12A) or Aspc-1 cancer cells (FIG. 12B) grown in 2D assays.

FIGS. 13A & 13B are bar graphs demonstrating SOF-367 and RAL-875 inhibit cell viability of Mia-Paca2 cancer cells (FIG. 13A) or Aspc-1 cancer cells (FIG. 13B) grown in 3D spheroids.

FIG. 14 illustrates the structure and the concentration-dependent percent inhibition of Rgl2-mediated guanine nucleotide exchange of RalB and RalB Tyr82Phe mutant after 24 h incubation at 4° C. with SOF 368.

FIG. 15 illustrates the structure and the concentration-dependent percent inhibition of Rgl2-mediated guanine nucleotide exchange of RalB and RalB Tyr82Phe mutant after 24 h incubation at 4° C. with SOF 369.

FIG. 16 illustrates the structure and the concentration-dependent percent inhibition of Rgl2-mediated guanine nucleotide exchange of RalB and RalB Tyr82Phe mutant after 24 h incubation at 4° C. with SOF 370.

FIG. 17 illustrates the structure and the concentration-dependent percent inhibition of Rgl2-mediated guanine nucleotide exchange of RalB and RalB Tyr82Phe mutant after 24 h incubation at 4° C. with SOF 371. SOF-371 show higher stability than Compound 1, and inhibits RalA and Ral B exchange. SOF371 inhibits Y82F suggesting higher affinity and non-covalent inhibition.

FIG. 18 illustrates the structure and the concentration-dependent percent inhibition of Rgl2-mediated guanine nucleotide exchange of RalB and RalB Tyr82Phe mutant after 24 h incubation at 4° C. with SOF 376.

FIG. 19 illustrates the structure of compounds SOF 377, SOF 378 and RLA-875.

FIG. 20 illustrates the structure and the concentration-dependent percent inhibition of Rgl2-mediated guanine nucleotide exchange of RalB and RalB Tyr82Phe mutant after 24 h incubation at 4° C. with SOF 379.

FIG. 21 illustrates the structure and the concentration-dependent percent inhibition of Rgl2-mediated guanine nucleotide exchange of RalB and RalB Tyr82Phe mutant after 24 h incubation at 4° C. with SOF 380.

FIG. 22 illustrates the structure and the concentration-dependent percent inhibition of Rgl2-mediated guanine nucleotide exchange of RalB and RalB Tyr82Phe mutant after 24 h incubation at 4° C. with SOF 381.

FIG. 23 illustrates the structure and the concentration-dependent percent inhibition of Rgl2-mediated guanine nucleotide exchange of RalB and RalB Tyr82Phe mutant after 24 h incubation at 4° C. with SOF 382.

FIG. 24 illustrates the structure and the concentration-dependent percent inhibition of Rgl2-mediated guanine nucleotide exchange of RalB and RalB Tyr82Phe mutant after 24 h incubation at 4° C. with SOF 531.

FIG. 25 illustrates the structure and the concentration-dependent percent inhibition of Rgl2-mediated guanine nucleotide exchange of RalB and RalB Tyr82Phe mutant after 24 h incubation at 4° C. with SOF 532.

FIG. 26 illustrates the structure and the concentration-dependent percent inhibition of Rgl2-mediated guanine nucleotide exchange of RalB and RalB Tyr82Phe mutant after 24 h incubation at 4° C. with SOF 533.

FIG. 27 illustrates the structure and the concentration-dependent percent inhibition of Rgl2-mediated guanine nucleotide exchange of RalB and RalB Tyr82Phe mutant after 24 h incubation at 4° C. with SOF 534.

FIG. 28 illustrates the structure and the concentration-dependent percent inhibition of Rgl2-mediated guanine nucleotide exchange of RalB and RalB Tyr82Phe mutant after 24 h incubation at 4° C. with SOF 535.

FIG. 29 illustrates the structure and the concentration-dependent percent inhibition of Rgl2-mediated guanine nucleotide exchange of RalB and RalB Tyr82Phe mutant after 24 h incubation at 4° C. with SOF 536.

### DETAILED DESCRIPTION

### Definitions

In describing and claiming the present disclosure, the following terminology will be used in accordance with the definitions set forth below.

As used herein, the term “pharmaceutically acceptable carrier” includes any of the standard pharmaceutical carriers, such as a phosphate buffered saline solution, water, emulsions such as an oil/water or water/oil emulsion, and various types of wetting agents. The term also encompasses any of the agents approved by a regulatory agency of the US Federal government or listed in the US Pharmacopeia for use in animals, including humans.

As used herein the term “pharmaceutically acceptable salt” refers to salts of compounds that retain the biological activity of the parent compound, and which are not biologically or otherwise undesirable. Many of the compounds disclosed herein are capable of forming acid and/or base salts by virtue of the presence of amino and/or carboxyl groups or groups similar thereto.

As used herein, the term “treating” includes prophylaxis of the specific disorder or condition, or alleviation of the symptoms associated with a specific disorder or condition and/or preventing or eliminating said symptoms.

As used herein an “effective” amount or a “therapeutically effective amount” refers to an alteration in the concentration of compound in a patient to provide a desired effect. For example one desired effect would be alleviating the symptoms associated with a disease state, wherein the disease state is aggravated by elevated levels of ADMA. In this embodiment the patient's blood or plasma would be contacted with a therapeutically effective amount of DDAH. The amount that is “effective” will vary from subject to subject, depending on the age and general condition of the individual, mode of administration, and the like. Thus, it is not always possible to specify an exact “effective amount.” However, an appropriate “effective” amount in any individual case may be determined by one of ordinary skill in the art using routine experimentation.

As used herein, the term “purified” and like terms relate to the isolation of a molecule or compound in a form that is substantially free of contaminants normally associated with the molecule or compound in a native or natural environment.

As used herein the term “patient” without further designation is intended to encompass any warm blooded vertebrate domesticated animal (including for example, but not limited to livestock, horses, mice, cats, dogs and other pets) and humans.

Each of the terms “about” and “approximately,” as used herein, mean greater or lesser than the value or range of values stated by 10 percent, but is not intended to designate any value or range of values to only this broader definition. Each value or range of values preceded by the term “about” or the term “approximately” also is intended to encompass the embodiment of the stated absolute value or range of values.

As used herein, the term “Rals” in the absence of further qualifications means a plurality of members of the Ral subfamily of GTPases. The Ras superfamily is a protein superfamily of GTPases that includes the Ras family. The Ras family includes six sub-families, two of which are the Ras subfamily and the Ral subfamily. Ral GTPases were discovered while searching for RAS-related genes. Two Ral GTPases have been identified, RalA and RalB. Like Ras, Ral GTPases cycle between an active GTP-bound and an inactive GDP-bound complex. GTP-bound Ral binds to a range of effector proteins triggering signaling through pathways that control multiple cellular processes. Ral effector proteins include RalBP1 (Ral binding protein 1)/RIP (Ral-interacting protein), Sec5, and exo84.

As used herein a Ral GTPAse driven tumor is a mass of cells that exhibit overexpression or inappropriate expression of a Ral GTPase.

As used herein, “administration” generally means prescription or provision of a pharmaceutical composition to a patient for self-administration by the patient, and may also mean direct administration of a pharmaceutical composition to a patient by a clinician.

The term “halogen” or variants such as “halide” or “halo” as used herein refers to fluorine, chlorine, bromine and iodine.

The terms “alkoxy,” “alkylamino” and “alkylthio” (or thioalkoxy) are used in their conventional sense, and refer to those alkyl groups attached to the remainder of the molecule via an oxygen atom, an amino group, or a sulfur atom, respectively.

The term “alkyl,” by itself or as part of another substituent, means, unless otherwise stated, a straight or branched chain, or cyclic hydrocarbon radical, or combination thereof, which may be fully saturated, mono- or polyunsaturated and can include di- and multivalent radicals, having the number of carbon atoms designated (i.e. C₁-C₁₀ means one to ten carbons).

The term “alkylene” by itself or as part of another substituent means a divalent radical derived from an alkane, as exemplified, but not limited, by the structures—CH₂CH₂— and —CH₂CH₂CH₂CH₂—, and further includes those groups described below as “heteroalkylene.” Typically, an alkyl (or alkylene) group will have from 1 to 24 carbon atoms, with those groups having 10 or fewer carbon atoms being a particular embodiment of the methods and compositions described herein. A “lower alkyl” or “lower alkylene” is a shorter chain alkyl or alkylene group, generally having eight or fewer carbon atoms.

The term “aryl” means, unless otherwise stated, a polyunsaturated, aromatic, hydrocarbon substituent which can be a single ring or multiple rings (including but not limited to, from 1 to 3 rings) which are fused together or linked covalently. The term “heteroaryl” refers to aryl groups (or rings) that contain from one to four heteroatoms selected from N, O, and S, wherein the nitrogen and sulfur atoms are optionally oxidized, and the nitrogen atom(s) are optionally quaternized. A heteroaryl group can be attached to the remainder of the molecule through a heteroatom. Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below.

### Embodiments

Cycling between GDP- to GTP-bound Ral is facilitated by guanine exchange factors (GEFs) and guanine activating proteins (GAPs) through a standard mechanism that is shared by members of the Ras superfamily. This process depends on the flexibility of two regions known as switch I (residues 41-51 in Ral GTPases) and switch II (residues 69-81 in Ral GTPases). GAPs catalyze the hydrolysis of GTP to GDP, while GEFs promote GDP to GTP exchange by favoring conformational states of Ral that favor GTP binding. Four of these Ral GEFs, namely RalGDS, Rgl1, Rgl2, and Rgl3, possess a Ras exchange motif (REM), a CDC25 homology domain, and a Ras association domain (RA). All four RalGEFs interact with active GTP-bound Ras through their RA domain, thereby directly activating Ral GTPases and making Ral-RalGEF a major Ras signaling pathway along with phosphoinositide 3-kinase (PI3K) and rapidly accelerated fibrosarcoma kinase (RAF).

Substantial evidence exists supporting a role for Ral GTPases in cancer that is both dependent and independent of Ras. As with Ras, there is great interest in small-molecule Ral antagonists for the development of cancer therapeutics. However, the development of small-molecule Ral antagonists has been very challenging. For example, Ral shares identical three-dimensional structure with Ras, which makes the specific targeting of Ral challenging. Moreover, both apo and complex structures of Ral and Ras GTPases are devoid of druggable pockets. The development of small molecules that bind reversibly to Ral or Ras has been achieved for solvent-exposed and shallow pockets, but none of these compounds engage Ral or Ras GTPases at therapeutic doses.

### Significance and Known Attempts by Others at Covalent Ras Inhibition

Although attempts have been made to identify small-molecule Ral and Ras antagonists for the development of cancer therapeutics, none of the known attempts have identified a compound that (1) engages Ral or Ras GTPases at therapeutic doses and (2) in a majority of Ral- or Ras-driven tumors.

As described in this section, a known covalent small-molecule Ras antagonist has been developed to inhibit Ras at therapeutic doses, prompted by the combination of a binding site and nucleophilic residue. For example, the K-Ras mutant G12C has enabled the development of a small-molecule covalent K-Ras inhibitor (e.g., AMG 510) that engages K-Ras G12C at therapeutic doses. This known small-molecule covalent Ras inhibitor works by targeting an accessible cysteine residue, such as the accessible cysteine residue available in K-Ras G12C. Covalent inhibitors overcome some drawbacks of small molecules that bind reversibly to Ral or Ras in that they do not require deep hydrophobic pockets to engage a target, as long as the reactive group of the covalent inhibitor can rapidly form a covalent bond with an amino acid side chain. Moreover, some covalent inhibitors have shown in vivo efficacy and are currently in clinical trials, despite showing low affinity for their targets. Historically, most FDA-approved covalent inhibitors fall into the category of mechanism-based inhibitors, which correspond to compounds that form a covalent bond with an enzyme active-site catalytic residue, or targeted covalent inhibitors, which form a covalent bond with bystander or non-catalytic residues. Most of the recently-approved covalent inhibitors, such as ibrutinib or afatibinib, along with investigational compounds like the K-Ras inhibitors AMG 510, MRTX849, and ARS-3248, are targeted covalent inhibitors that form a covalent bond at cysteine.

One significant drawback to known covalent inhibitors, such as AMG 510, is that all Ral proteins, as well as a great majority of Ras mutants such as oncogenic K-Ras, are devoid of a druggable pocket and lack an accessible cysteine residue that is amenable to covalent bonding with a small-molecule inhibitor. Indeed, the rare K-Ras mutant (G12C) only occurs in about 11-16% of lung adenocarcinomas and about 1-4% of pancreatic and colorectal adenocarcinomas. Despite its efficacy, the applicability of the known small-molecule inhibitor that targets K-Ras G12C is limited to Ras proteins having an accessible cysteine residue, such as K-Ras G12C, and thus is not indicated for the vast majority of Ras- or Ral-driven tumors.

These observations do not suggest a covalent Ral inhibitor that will be indicated for a vast majority of Ral-driven tumors. Instead, the known attempts have been directed to residues accessible in particular mutants. Hence, new strategies to develop covalent inhibitors are needed. Described below are embodiments of a screening method for identifying such covalent inhibitors, and their targets. For example, as discussed below, it was discovered that a tyrosine residue on Ral and Ras GTPases can be modified with a covalent inhibitor. This discovery could have profound implications for Ral and Ras drug discovery since the tyrosine is present in both proteins.

### Embodiments

Several reactive groups may be usable for covalent bond formation at residues other than cysteine. Examples include S(VI)-containing groups sulfonyl fluorides, which react at tyrosine, lysine or serine residues. Aryl sulfonyl fluorides provide useful tools to (i) identify amino acids that are amenable to covalent bond formation; (ii) uncover new pockets that can be used in drug development; (iii) provide starting points to develop derivatives with higher affinity and more suitable reactive groups. There are several tyrosine, lysine, and serine residues on Ral GTPases located at the interface between Ral GTPases and their GEFs or effector proteins. Among them is Tyr-82, which in K-Ras is equivalent to Tyr-71, is located near pockets that are the binding site of fragment and small molecules on Ral and Ras.

In one embodiment, covalent bond formation by an aryl sulfonyl fluoride electrophile at a tyrosine residue (Tyr-82) inhibits guanine exchange factor Rgl2-mediated nucleotide exchange of Ral GTPase. Screening of a covalent fragment library containing aryl sulfonyl fluorides led to the discovery of a class of such compounds that forms a covalent bond with non-catalytic residue Tyr-82. A high-resolution 1.18-Å X-ray co-crystal structure shows that the compound binds to a new well-defined druggable binding site in RalA as a result of a switch II loop conformational change. This druggable binding site is a deep hydrophobic pocket that was never previously observed in Ras or Ral GTPases. This binding pocket has a SiteMap DrugScore (druggability score) that is identical to druggable ATP-binding pockets on kinases, suggesting that it could be used to develop therapeutics targeting oncogenic Ras lacking cysteine.

The structures of such compounds that form a covalent bond with non-catalytic residue Tyr-82, along with additional high-resolution crystal structures of several analogs in complex with RalA, confirm the importance of key hydrogen bond anchors between compound sulfone oxygen atoms and Ral backbone nitrogen atoms. This newly-discovered druggable pocket and the covalent modification of a bystander tyrosine residue present in Ral and Ras GTPases provide a new strategy for developing therapeutic agents targeting oncogenic Ras mutants that are devoid of a cysteine nucleophile.
In accordance with one embodiment a compound is provided having the general structure of

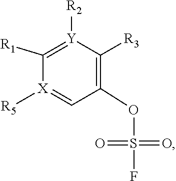

wherein
R₁ is H, ═O, —OCH₃, —OCH₂CH₃, —COCH₃,

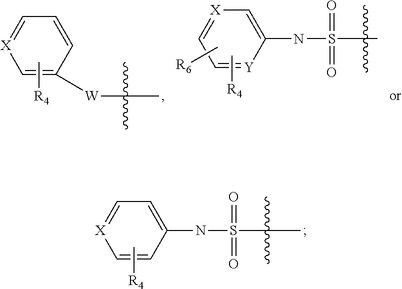

R₂ is H, —OCH₃, —OCH₂CH₃, C₁-C₄ alkyl, or CF₃;
R₃ is H, —OCH₃, —OCH₂CH₃, C₁-C₄ alkyl, CH₂(morpholino) or R₂ and R₃ together with the atom to which they are attached form a 5 to 6 membered cyclic, 5 to 6 membered heterocyclic, or 5 to 6 membered aryl ring, optionally forming a 1,4 dioxane, cyclohexane, morpholino, or piperazinyl ring;
R₄ is H, C₁-C₄ alkyl, —OCH₃, —OCH₂CH₃, —OCH(CH₃)₂, —(SO₂)CH₃, —OH, or halo;
R₅ is H or

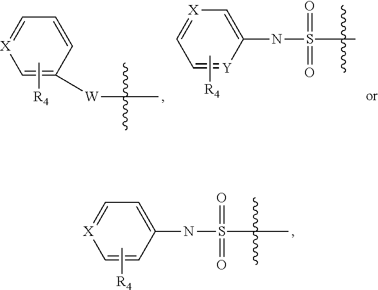

with the

proviso that R₁ and R₅ are not both H and one of R₁ and R₅ is other than

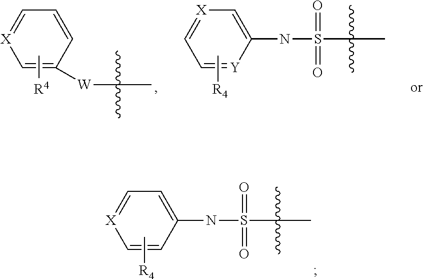

or R₁ and R₅ together with the atoms to which they are attached form a 5 to 6 membered cycloalkyl, 5 to 6 membered heterocyclic, or 5 to 6 membered aryl ring, optionally forming a 1,4 dioxane, cyclohexane, benzene, or piperazinyl ring;
R₆ is H or halo;
X and Y are independently C or N; and
W is

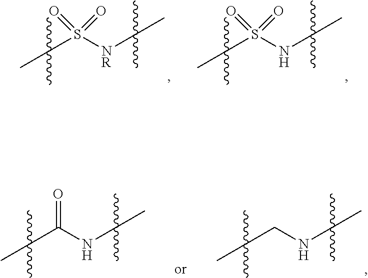

wherein R is H or C₁-C₄ alkyl. In a further embodiment
R₁ is H or ═O;
R₂ is H, C₁-C₄ alkyl, or CF₃;
R₃ is H or R₂ and R₃ together with the atom to which they are attached form a 6 membered heterocyclic, aryl ring, optionally a 1,4 dioxane, cyclohexane, benzene, morpholino, or piperazinyl ring;
R₄ is H, C₁-C₄ alkyl, —OCH₃, —OCH₂CH₃, —OCH(CH₃)₂, —(SO₂)CH₃, —OH, or halo;

R₅ is

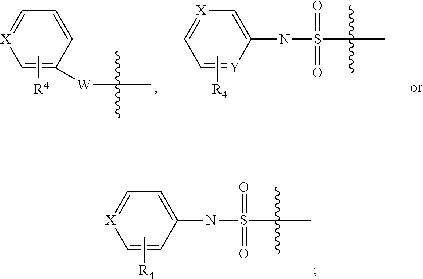

X and Y are independently C or N, optionally with the proviso that X and Y are not both N and
W is

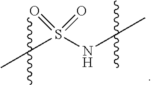 [SMILES: *NS(*)(=O)=O]

In accordance with one embodiment an aryl sulfonyl fluoride suitable for use in accordance with the present disclosure is provided having the structure of Formula I:

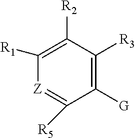

wherein

G is

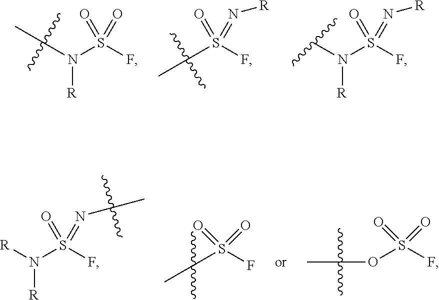

wherein R is H or C₁-C₄ alkyl, optionally wherein G is

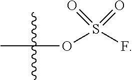 [SMILES: *OS(=O)(=O)F]

R₁ is H,

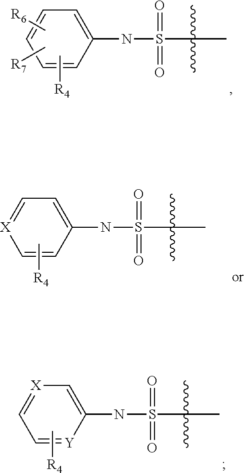

R₂ is H, C₁-C₄ alkyl, or CF₃,

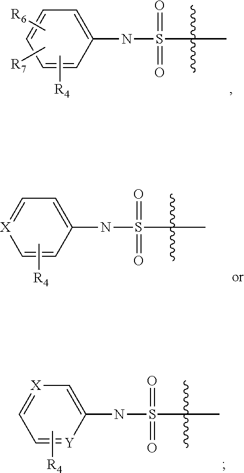

with the proviso that only one of R₁ or R₂ is

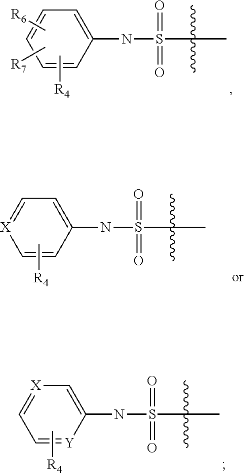

R₃ is H, C₁-C₄ alkyl, —OCH₃, —OCH₂CH₃, —OCH(CH₃)₂, or R₂ and R₃ together with the atom to which they are attached form a 6 membered heterocyclic, or a 6 membered aryl ring, optionally a 1,4 dioxane or hexacyclic ring;

R₄ is C₁-C₄ alkyl, —OCH₃, —OCH₂CH₃, —OCH(CH₃)₂, —(SO₂)CH₃, —OH, or halo;

R₅ is H or ═O;

R₆ and R₇ are independently halo;

X, Y and Z are independently C or N, optionally wherein Z is N and X and Y are each C, optionally wherein X is N and Y and Z are each C and optionally wherein X, Y and Z are each C.

In accordance with one embodiment an aryl sulfonyl fluoride suitable for use in accordance with the present disclosure is provided having the structure of

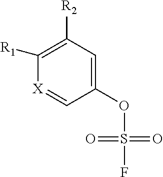

wherein

R₁ and R₂ are independently H or

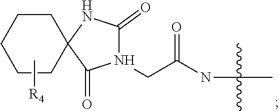

with the proviso that one of R₁ or R₂ is H;

R₄ is C₁-C₄ alkyl, —OCH₃, —OCH₂CH₃, —OCH(CH₃)₂, —(SO₂)CH₃, —OH, or halo, optionally wherein R₄ is C₁-C₂ alkyl or —OCH₃; and

X is C or N.

In one embodiment an aryl sulfonyl fluoride suitable for use in accordance with the present disclosure is provided having the structure of:

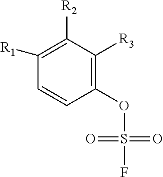

wherein

R₁ is

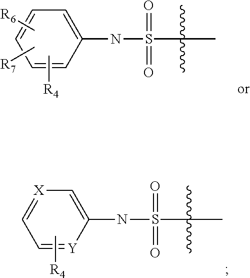

R₃ is H, C₁-C₄ alkyl, —OCH₃, —OCH₂CH₃, —OCH(CH₃)₂;

R₂ is H, or R₂ and R₃ together with the atom to which they are attached form a 6 membered aryl ring, a 1,4 dioxane or hexacyclic ring;

R₄ is C₁-C₄ alkyl, —OCH₃, —OCH₂CH₃, —OCH(CH₃)₂, —(SO₂)CH₃, —OH, or halo;

R₆ and R₇ are independently halo or H, optionally wherein said halo is Cl or F;

X and Y are independently C or N, optionally wherein X is N and Y is C.

In one embodiment an aryl sulfonyl fluoride suitable for use in accordance with the present disclosure is provided having the structure of:

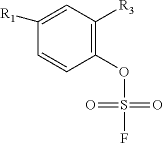

wherein

R₁ is

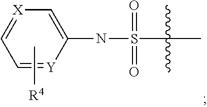

R₃ is H, C₁-C₄ alkyl, —OCH₃, —OCH₂CH₃, or —OCH(CH₃)₂;

R₄ is —OCH₃, —OCH₂CH₃, —OCH(CH₃)₂, —OH, or halo;

X and Y are independently C or N, optionally wherein X is N and Y is C.

In one embodiment an aryl sulfonyl fluoride suitable for use in accordance with the present disclosure is provided having the structure of:

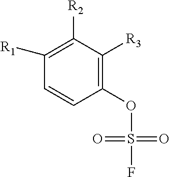

wherein

R₁ is

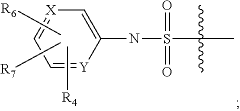

R₂ and R₃ together with the atom to which they are attached form a 1,4 dioxane or hexacyclic ring;

R₄ is —OCH₃, —OCH₂CH₃, —OCH(CH₃)₂, or —OH;

R₆ and R₇ are independently H or halo, optionally wherein said halo is F or Cl, and

X and Y are independently C or N, optionally wherein X is N and Y is C. In a further embodiment R₆ is H, X is N and Y is C.

In an embodiment, the small-molecule compound that forms a covalent bond with a residue of Ral, thereby inhibiting Ral, is any of Compounds 1-20 of Table 1.

In an embodiment, a method of inhibiting a Ral GTPase and/or treating a patient having a cancer characterized by a mutant Ral GTPase includes administering a compound having the structure of:

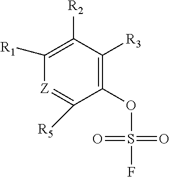

wherein

R₁ is H,

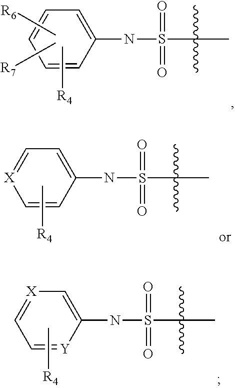

R₂ is H, C₁-C₄ alkyl, or CF₃,

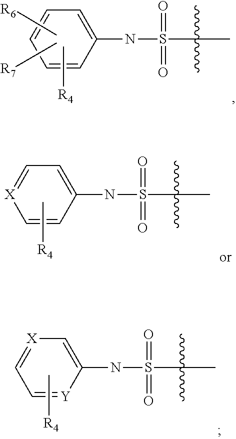

with the proviso that only one of R₁ or R₂ is

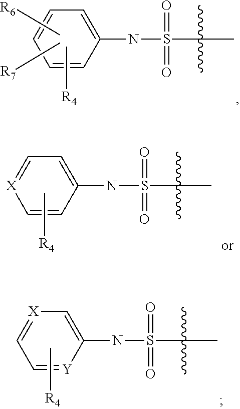

R₃ is H, C₁-C₄ alkyl, —OCH₃, —OCH₂CH₃, —OCH(CH₃)₂, or R₂ and R₃ together with the atom to which they are attached form a 6 membered heterocyclic, or a 6 membered aryl ring, optionally a 1,4 dioxane or hexacyclic ring;

R₄ is C₁-C₄ alkyl, —OCH₃, —OCH₂CH₃, —OCH(CH₃)₂, —(SO₂)CH₃, —OH, or halo;

R₅ is H or ═O;

R₆ and R₇ are independently halo;

X, Y and Z are independently C or N, optionally wherein Z is N and X and Y are each C, optionally wherein X is N and Y and Z are each C and optionally wherein X, Y and Z are each C.

In one embodiment, a method of inhibiting a Ral GTPase and/or treating a patient having a cancer characterized by a mutant Ral GTPase includes administering a compound having the structure of:

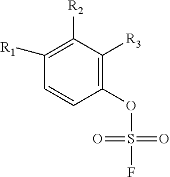

wherein

R₁ is

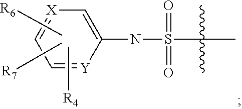

R₂ and R₃ together with the atom to which they are attached form a 1,4 dioxane or hexacyclic ring;

R₄ is —OCH₃, —OCH₂CH₃, —OCH(CH₃)₂, or —OH;

R₆ and R₇ are independently H or halo, optionally wherein said halo is F or Cl, and

X and Y are independently C or N, optionally wherein X is N and Y is C. In a further embodiment R₆ is H, X is N and Y is C.

In one embodiment, a method of inhibiting a Ral GTPase and/or treating a patient having a cancer characterized by a mutant Ral GTPase includes administering a compound having the structure of:

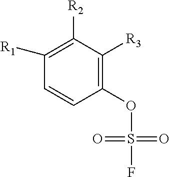

wherein

R₁ is

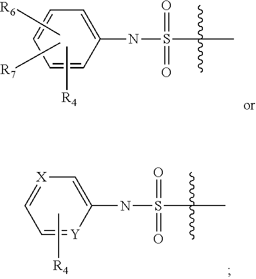

15

R₃ is H, C₁-C₄ alkyl, —OCH₃, —OCH₂CH₃, —OCH(CH₃)₂;

R₂ is H, or R₂ and R₃ together with the atom to which they are attached form a 6 membered aryl ring, a 1,4 dioxane or hexacyclic ring;

R₄ is C₁-C₄ alkyl, —OCH₃, —OCH₂CH₃, —OCH(CH₃)₂, —(SO₂)CH₃, —OH, or halo;

R₆ and R₇ are independently halo or H, optionally wherein said halo is Cl or F;

X and Y are independently C or N, optionally wherein X is N and Y is C.

In one embodiment, a method of inhibiting a Ral GTPase and/or treating a patient or having a cancer characterized by a mutant Ral GTPase includes administering one or more compounds selected from the group consisting of

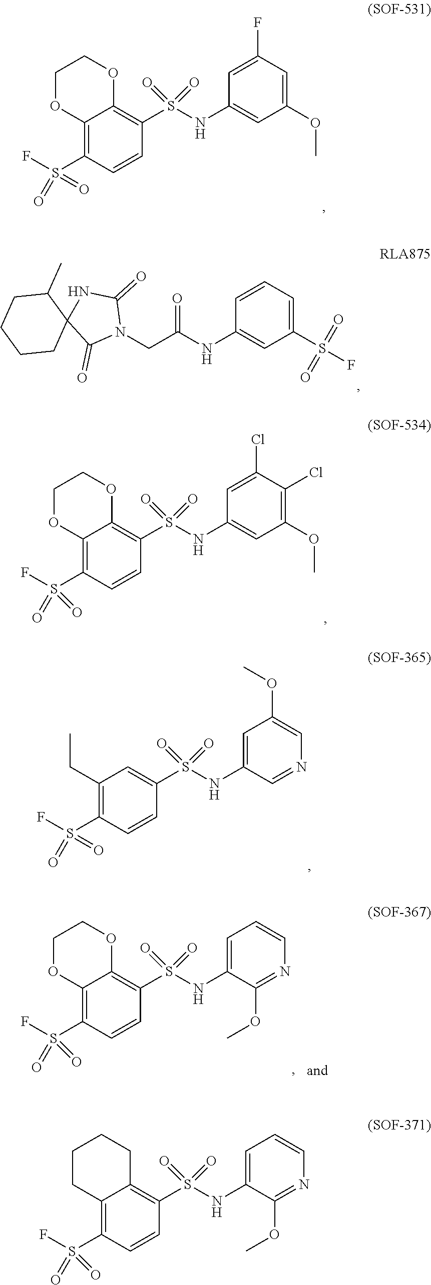 [SMILES: COc1cc(F)cc(NS(=O)(=O)c2ccc(S(=O)(=O)F)c3c2OCCO3)c1 ; CC1CCCCC12NC(=O)N(CC(=O)Nc1cccc(S(=O)(=O)F)c1)C2=O ; COc1cc(NS(=O)(=O)c2ccc(S(=O)(=O)F)c3c2OCCO3)cc(Cl)c1Cl ; CCc1cc(S(=O)(=O)Nc2cncc(OC)c2)ccc1S(=O)(=O)F ; COc1ncccc1NS(=O)(=O)c1ccc(S(=O)(=O)F)c2c1OCCO2 ; COc1ncccc1NS(=O)(=O)c1ccc(S(=O)(=O)F)c2c1CCCC2]

In one embodiment any of the the sulfonyl fluoride groups of the compounds disclosed herein can be substituted with a derivative group selected from

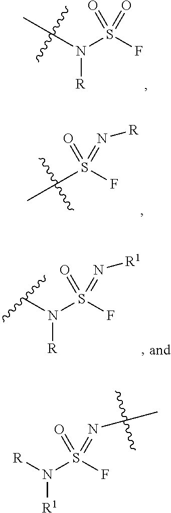

wherein R and R¹ are independently H or C₁-C₄ alkyl.

In an embodiment, a method of inhibiting a Ral GTPase and/or treating a patient or having a cancer characterized by a mutant Ral GTPase includes administering at least one of the compounds listed in Table 1.

TABLE 1
Compound Chemical Structures

| | 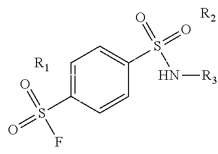 | | | IC₅₀ at 24 h (μM) | |
|---|---|---|---|---|---|
| Compound | R₁ | R₂ | R₃ | RalB WT | RalB Tyr82Phe |
| 1 | 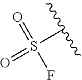 [SMILES: *S(=O)(=O)F] | 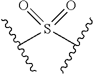 [SMILES: *S(*)(=O)=O] | 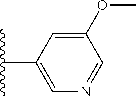 [SMILES: *c1cncc(OC)c1] | 49.5 ± 2.3 | NI* |
| 2 | 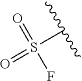 [SMILES: *S(=O)(=O)F] | 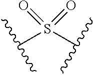 [SMILES: *S(*)(=O)=O] | 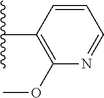 [SMILES: *c1cccnc1OC] | 23.1 ± 6.8 | NI |
| 3 | 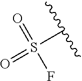 [SMILES: *S(=O)(=O)F] | 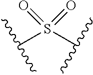 [SMILES: *S(*)(=O)=O] | 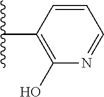 [SMILES: *c1cccnc1O] | 17.0 ± 7.7 | 79.3 ± 19.0 |
| 4 | 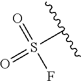 [SMILES: *S(=O)(=O)F] | 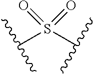 [SMILES: *S(*)(=O)=O] | 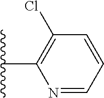 [SMILES: *c1ncccc1Cl] | 41.7 ± 8.5 | 215 ± 84.5 |
| 5 | 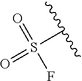 [SMILES: *S(=O)(=O)F] | 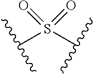 [SMILES: *S(*)(=O)=O] | 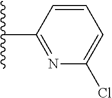 [SMILES: *c1cccc(Cl)n1] | 24.4 ± 4.9 | NI |
| 6 | 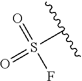 [SMILES: *S(=O)(=O)F] | 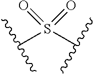 [SMILES: *S(*)(=O)=O] | 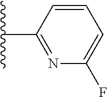 [SMILES: *c1cccc(F)n1] | 51.5 ± 5.2 | NI |
| 7 | 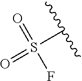 [SMILES: *S(=O)(=O)F] | 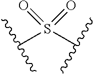 [SMILES: *S(*)(=O)=O] | 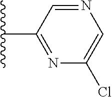 [SMILES: *c1cncc(Cl)n1] | 49.4 ± 3.5 | NI |
| 8 | 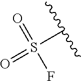 [SMILES: *S(=O)(=O)F] | 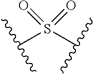 [SMILES: *S(*)(=O)=O] | 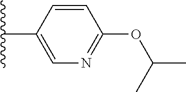 [SMILES: *c1ccc(OC(C)C)nc1] | ND† | ND |
| 9 | 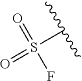 [SMILES: *S(=O)(=O)F] | 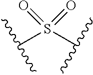 [SMILES: *S(*)(=O)=O] | 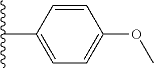 [SMILES: *c1ccc(OC)cc1] | 60.3 ± 3.0 | 100 ± 11.1 |
| 10 | 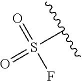 [SMILES: *S(=O)(=O)F] | 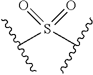 [SMILES: *S(*)(=O)=O] | 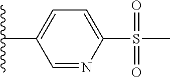 [SMILES: *c1ccc(S(C)(=O)=O)nc1] | 147 ± 42.0 | 238 ± 55.4 |
| 11 | 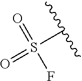 [SMILES: *S(=O)(=O)F] | 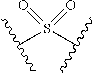 [SMILES: *S(*)(=O)=O] | 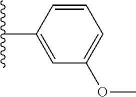 [SMILES: *c1cccc(OC)c1] | 26.0 ± 4.6 | 164 ± 42.3 |
| 12 | 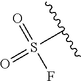 [SMILES: *S(=O)(=O)F] | 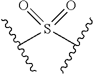 [SMILES: *S(*)(=O)=O] | 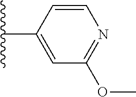 [SMILES: *c1ccnc(OC)c1] | 146 ± 14.2 | NI |
| 13 | 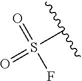 [SMILES: *S(=O)(=O)F] | 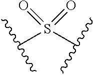 [SMILES: *S(*)(=O)=O] | 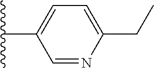 [SMILES: *c1ccc(CC)nc1] | 164 ± 26.0 | NI |
| 14 | 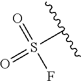 [SMILES: *S(=O)(=O)F] | 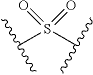 [SMILES: *S(*)(=O)=O] | 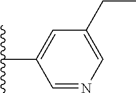 [SMILES: *c1cncc(CC)c1] | 91.4 ± 2.9 | 167 ± 8.7 |
| 15 | 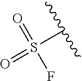 [SMILES: *S(=O)(=O)F] | 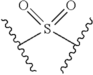 [SMILES: *S(*)(=O)=O] | 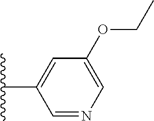 [SMILES: *c1cncc(OCC)c1] | 85.5 ± 7.9 | 235 ± 167 |
| 16 | 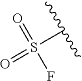 [SMILES: *S(=O)(=O)F] | 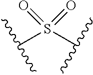 [SMILES: *S(*)(=O)=O] | 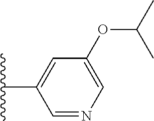 [SMILES: *c1cncc(OC(C)C)c1] | 79.5 ± 2.3 | 333 ± 31.0 |
| 17 | 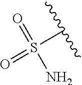 [SMILES: *S(N)(=O)=O] | 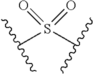 [SMILES: *S(*)(=O)=O] | 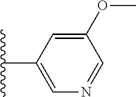 [SMILES: *c1cncc(OC)c1] | ND | ND |
| 18 | 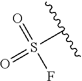 [SMILES: *S(=O)(=O)F] | 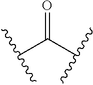 [SMILES: *C(*)=O] | 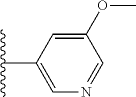 [SMILES: *c1cncc(OC)c1] | ND | ND |
| 19 | 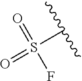 [SMILES: *S(=O)(=O)F] | 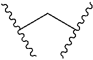 [SMILES: *C*] | 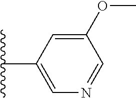 [SMILES: *c1cncc(OC)c1] | ND | ND |
| 20 | 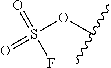 [SMILES: *OS(=O)(=O)F] | 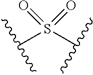 [SMILES: *S(*)(=O)=O] | 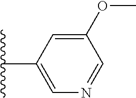 [SMILES: *c1cncc(OC)c1] | ND | ND |
| 21 | 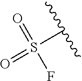 [SMILES: *S(=O)(=O)F] | 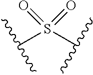 [SMILES: *S(*)(=O)=O] | 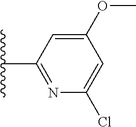 [SMILES: *c1cc(OC)cc(Cl)n1] | 24.0 ± 1.0 | 138 ± 34.2 |
| 22 | 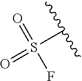 [SMILES: *S(=O)(=O)F] | 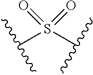 [SMILES: *S(*)(=O)=O] | 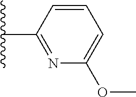 [SMILES: *c1cccc(OC)n1] | 64.0 ± 4.1 | NI |
| 23 | 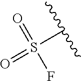 [SMILES: *S(=O)(=O)F] | 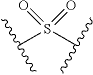 [SMILES: *S(*)(=O)=O] | 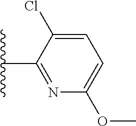 [SMILES: *c1nc(OC)ccc1Cl] | 127 ± 11.7 | NI |

*No Inhibition
†Not Determined

An application of an embodiment of a method of screening a library of small-molecule compounds is discussed below in accordance with this disclosure. This screening method enables evaluation of the suitability of a residue of interest for the development of Ral covalent inhibitors. In the embodiment, discussed below, the small molecule compounds are aryl sulfonyl fluoride fragments and the residue is Tyr-82 of Ral GTPases. However, such methods are not limited to such compounds and residues. This screening method also enables preparation of several derivatives along with additional high-resolution structures afforded a limited structure-activity relationship study further confirming the existence of the pocket and the importance of key hydrogen bonding interactions.

### Example Screening Method According to an Embodiment of this Disclosure

Small Molecules Inhibit Ral Nucleotide Exchange by Rgl2: Analysis of the three-dimensional NMR structures of RalB in complex with Ral effector protein RalBP1 (PDB code: 2KWI) shows the presence of a shallow but well-defined binding site occupied by RalBP1 Trp-430. In addition, the structure of Ral in complex with a Ral guanine exchange factor Rgl2 (PDB code: 5CM8) shows that this binding site is located at the Ral⋅Rgl2 protein-protein interface. Although the binding site is well-defined, it is not sufficiently deep and hydrophobic to accommodate a small molecule that can engage Ral at therapeutic doses. As a result, the possibility of developing a covalent inhibitor of Rgl2 Ral activation was investigated. Although there are no accessible cysteine residues on Ral, there exists a tyrosine (Tyr-82) near the Trp-430 binding site that could provide an opportunity for covalent bond formation with an electrophile.

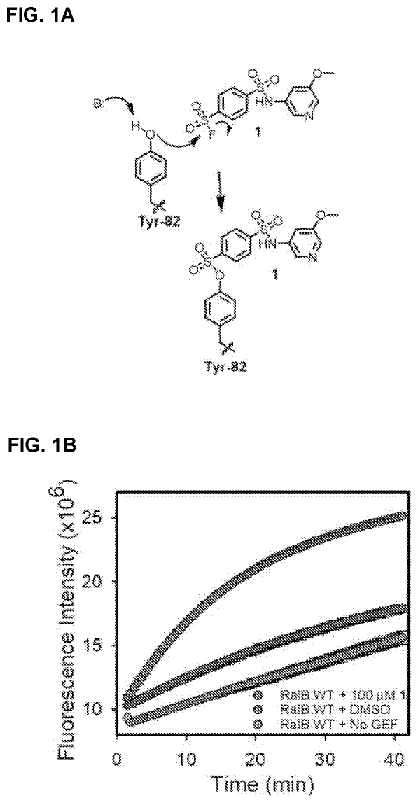

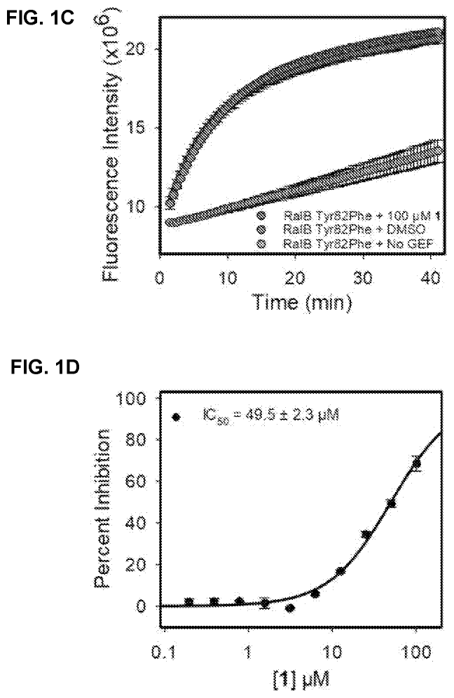

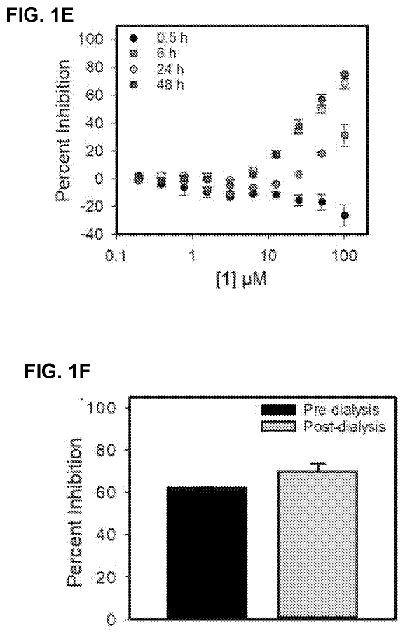

Fragment-based screening using a library of 89 sulfonyl fluoride compounds was carried out to explore this possibility. A fluorescence-based guanine nucleotide exchange assay was used to measure inhibition of Rgl2-mediated exchange of Ral-bound GDP with fluorescently-labelled boron-dipyrromethene fluorescent GDP (BODIPY-FL-GDP). The increase in fluorescence intensity of the BODIPY-FL group is measured at 30 s intervals. The exchange is initiated by the addition of Rgl2 and BODIPY-FL-GDP after the compound has been pre-incubated with Ral. Compound 1, illustrated in FIG. 1A, was identified to inhibit the Rgl2-mediated nucleotide exchange of RalB. This outcome is illustrated in FIG. 1B, which shows percent inhibition as a function of the concentration of the compound.

To confirm that inhibition of RalB exchange by Rgl2 was due to covalent bond formation at Tyr-82, the nucleotide exchange study was repeated using RalB Tyr82Phe mutant. The RalB Tyr82Phe mutant showed robust nucleotide exchange by Rgl2. This outcome is illustrated in FIG. 1C, which shows that the compound completely lost its ability to inhibit Rgl2 nucleotide exchange of RalB Tyr82Phe mutant. A concentration-dependent exchange study then was carried out to obtain the concentration of compound required for 50% inhibition of Rgl2-mediated exchange, as illustrated in FIG. 1D. This was done by incubating RalB with varying concentrations of Compound 1 for 24 hours at a temperature of 4° C., prior to the initiation of nucleotide exchange by the addition of Rgl2 and BODIPY-FL-GDP. The rate constant was calculated by fitting a 3-parameter exponential function for each measurement. To determine percent inhibition, the fitting was also done for control DMSO samples and samples without Rgl2. A plot of percent inhibition versus compound concentration resulted in an IC₅₀ of 49.5±2.3 μM following 24 h incubation at 4° C. is shown in FIG. 1D.

To further establish that the compound is a covalent inhibitor, a time-dependent study for Rgl2-mediated exchange of RalB nucleotide was carried out. The outcome of this study is illustrated in the chart of FIG. 1E. At 30 mins, there was no inhibition of exchange detected at the range of concentrations that were considered for the compound. At 6 h, the compound inhibited exchange with an IC₅₀ of 153±29.9 μM. At 24 h, even greater inhibition of RalB nucleotide exchange of Rgl2 was observed. No further increase in the extent of inhibition of the compound was found to occur from 24 to 48 h. The time-dependent inhibition of exchange confirms that Compound 1 is a covalent inhibitor of RalB activation by Rgl2.

Sulfonyl fluorides are considered irreversible inhibitors. The tyrosine oxygen is expected to form a covalent bond with the sulfur atom of the compound displacing the fluorine atom in a substitution reaction as illustrated in FIG. 1A. Protein dialysis was used to establish that the inhibition of RalB by Compound 1 is irreversible. RalB was incubated with compound for 24 h at 4° C., followed by 24 h dialysis at 4° C. to remove the presence of excess compound. As shown in the chart of FIG. 1F, nucleotide exchange of RalB by Rgl2 was completely inhibited despite the absence of excess compound in solution confirming that the compound is an irreversible covalent inhibitor of RalB.

Intact (i.e., whole) protein mass spectrometry was used to further establish that the compound forms a covalent bond with RalB at Tyr-82. Following incubation of RalB with 50 μM Compound 1 for 12 h at 4° C., a peak at m/z 24219 was observed that corresponds to the RalB protein. Another peak at m/z 24545 (RalB+326) corresponds to the adduct formation by, which has a molecular weight of 346 g/mol, and the adduct reflects the fact that a Fluorine atom from the compound and a Hydrogen atom from the protein have been eliminated. A small secondary peak at m/z 24872 (RalB+653) was observed, which equates to two simultaneous adducts, indicating a secondary reaction site. The same experiment was repeated except that RalB Tyr82Phe was incubated with Compound 1 for 24 h at 4° C. The spectrum shows a peak at m/z 24203 (RalB^{WT}−16) corresponding to RalB Tyr82Phe. Due to the loss of reaction at Tyr-82, only a minor peak is observed at m/z 24529 (RalB^{Tyr82Phe}+326). These results further confirm that Compound 1 forms a covalent adduct with RalB at Tyr-82.

High Resolution Crystal Structures of Covalent Complex Reveals Druggable Pocket. X-ray crystallography was used to determine the binding mode of Compound 1 with Ral GTPase. Attempts to crystallize RalB.GDP did not yield quality crystals, while RalA.GDP readily crystallized to yield the first X-ray structure of a human Ral GTPase structure. Since there is high sequence identity between RalA and RalB, GDP-bound RalA crystals were soaked with Compound 1, which led to a high resolution 1.18-Å structure of the covalent complex. In addition to revealing the binding mode of Compound 1, the clear electron density confirmed the presence of a covalent bond between the sulfone sulfur atom of Compound 1 and the hydroxyl oxygen of Tyr-82 further establishing the existence of the covalent RalA-Compound 1 (RalA-1) complex at Tyr-82. Remarkably, the compound created a new well-defined and deep binding site within RalA. This binding site is not present in any crystal structure of apo Ras or Ral GTPases or in complexes of these proteins with fragments and compounds.

In addition to the RalA-1 complex, two high-resolution crystal structures of human apo RalA were solved. These structures highlight the flexibility of the switch II loop, where the segment Ala-70-Tyr-75 is present in more than one conformation.

Data were collected from three different crystals (crystal 1 at resolution 1.55 Å, crystal 2 at 1.54 Å [PBD ID: 6P0O] and crystal 3 at 1.31 Å [PDB ID: 6P0J]) to confirm this observation. In the three crystals, the electron density around the Ala-70-Tyr-75 loop presents as at least two distinct conformations (“open” and “closed”) and are fitted for individual data sets. The “open” conformation of the loop (PDB ID: 6P0O) is similar to the RalA-1 complex, except for Glu-73, which is flipped into the binding pocket in the apo structure. The “closed” conformation of the loop (PDB ID: 6P0J) is significantly different from the RalA-1 complex. The Schrödinger SiteMap program was used to determine the druggability of the pocket occupied by Compound 1. In the apo RalA structures, the volume of the pocket ranges from 150 Å³ (PDB ID: 1U8Y) to 187 Å³ (PDB ID: 6P0O). In the RalA-1 complex, the pocket has a volume of 221 Å³. The SiteMap program also provides measures to assess ligand binding and druggability of a pocket known as SiteScore and DrugScore, respectively. These scores are calculated using the hydrophobicity and accessibility of a detected binding site. Unlike DrugScore, SiteScore limits the impact of hydrophilicity in charged and highly polar sites. A DrugScore of 1 or above suggests that a pocket is druggable. The ATP-binding pocket of kinases, which is the active site of many FDA approved drugs, have DrugScore greater than Compound 1. For example, the ATP-binding pocket of CDK6 bound to the FDA approved drug abemaciclib (PDB ID: 5L2S) is 1.1. Another druggable pocket is the acetylated lysine recognition site on bromodomains. One example is the druggable pocket of the bromodomain BRD4 occupied by CPI-0610 (PDB ID: 5HLS), a compound currently in clinical trials, has a DrugScore of 1.08. Similarly, the pocket on RalA that is occupied by Compound 1 has a DrugScore of 1.04, suggesting that this pocket is also druggable.

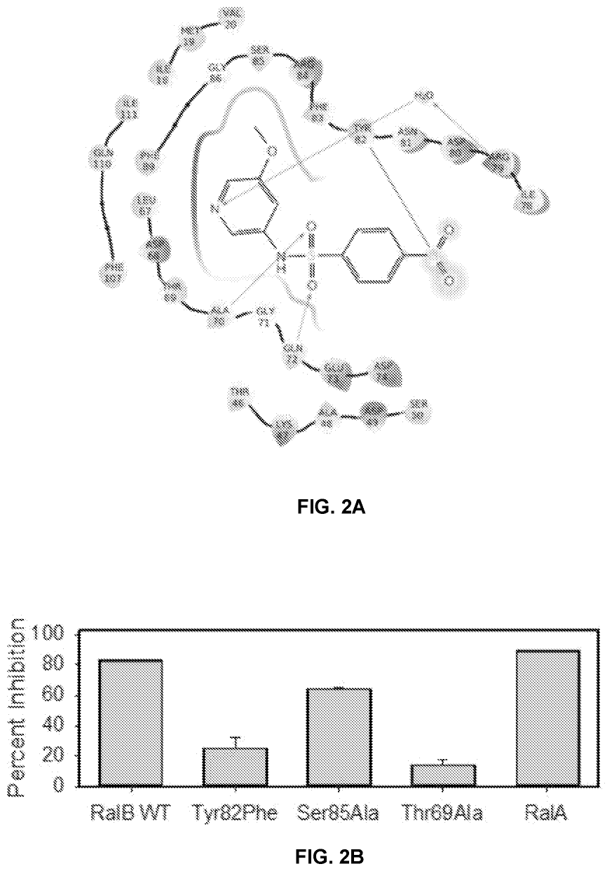

The RalA-1 structure shows that the compound is anchored by two hydrogen bond interactions between each of its sulfonamide oxygen atoms and backbone amide nitrogen atoms of Ala-70 and Gln-72. These two residues are located on the flexible switch II loop region. In the open conformation structure of apo RalA, the backbone nitrogen atoms of Ala-70 and Gln-72 are well positioned to donate to the hydrogen bonds, indicating that the pocket is partially primed for Compound 1. The Glu-73 residue is flipped out of the binding pocket to make room for the compound. Interestingly, the methoxy group of Compound 1 is located in a region that is occupied by the side chain of Phe-83. In the RalA-1 complex, the Phe-83 side chain rotates from its native orientation that is seen in the apo structure to accommodate the methoxy group of Compound 1. The nitrogen atom of the pyridine ring of Compound 1 forms a water-mediated hydrogen bond with the guanidinium ion of Arg-79. Finally, the compound engages several hydrophobic residues, including Ile-18, Val-20, Ala-48, Leu-67, and Phe-83, through van der Waals interactions, as illustrated in FIG. 2A.

Considering that the structure of Compound 1 was solved with RalA, an exchange study was carried out to confirm that Compound 1 also inhibits RalA, which is identical in overall structure to RalB and possess more than 80% sequence similarity. As shown in FIG. 2B, Compound 1 inhibited RalA and RalB to the same extent. To further probe the contribution of individual amino acids within the binding site of Compound 1, the effect of the compound on the rate of nucleotide exchange was tested against two RalB mutants, Ser85Ala and Thr69Ala. Thr-69 comes in direct contact with the compound, while Ser-85 is located in the vicinity but does not come in contact with the compound. As expected, the Ser85Ala mutation did not affect the inhibition of RalB by Compound 1. Thr69Ala mutation, on the other hand, which is within the binding pocket, substantially impaired the ability of the compound to inhibit RalB exchange, as illustrated in FIG. 2B.

Compound 1 Selectively Inhibits Ral over Ras: Ral and Ras GTPases have similar three-dimensional structures. Multiple sequence alignment of RalA and RalB to K-Ras as well as representative members of other GTPases in the Ras superfamily reveal similarities in the amino acid composition of the binding site of Compound 1 (AA). Superimposition of our RalA-1 complex with the structure of K-Ras shows that K-Ras, like Ral GTPases, possesses a tyrosine residue (Tyr-71) at the same position occupied by Ral Tyr-82.

A sequence alignment between Ras subfamily members demonstrated that this tyrosine is present in nearly all the Ras superfamily GTPases, except for RhoA and Rac1 in the Rho subfamily. Residues numbers are in reference to their respective position on RalA. Multiple sequence alignment was performed using Clustal Omega (v1.2.4). The presence of a tyrosine at position 82 suggests that Compound 1 should form a covalent bond with K-Ras Tyr-71 and inhibit GEF nucleotide exchange of the GTPase. However, the structures also reveal some differences, such as the presence of a glutamic acid on K-Ras (Glu-37) instead of an alanine residue on Ral at the same position (Ala-48). Whether Compound 1 inhibited SOS-catalyzed nucleotide exchange of K-Ras was tested using a similar fluorescence-based guanine nucleotide exchange assay that was developed for Ral. Compound 1 did not inhibit the SOS-mediated guanine-nucleotide exchange of K-Ras.

Further examination of K-Ras crystal structures reveals the presence of a hydrogen bond between Glu-37 and the backbone nitrogen of Ala-59. As K-Ras Ala-59 is the equivalent of Ral Ala-70, and the interaction between the sulfone oxygen of Compound 1 and the backbone of Ral Ala-70 is critical, the Glu-37 hydrogen bond may further reduce the ability of Compound 1 to bind to this pocket on K-Ras. These results suggest that Glu-37, which protrudes into the binding site of Compound 1, may block access to the druggable pocket on K-Ras by Compound 1. Sequence alignment of representative Ras superfamily members indicates that glutamic acid or phenylalanine are prevalent at this position, while several other members have a glycine residue.

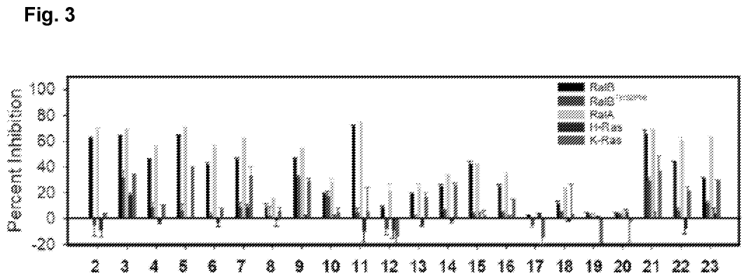

Compound 1 Derivatives and Crystal Structures Confirm Covalent Complex and Binding Site: Several derivatives of Compound 1 were prepared, as illustrated in Table 1. The compounds were tested against RalB wild-type, RalB Tyr82Phe mutant, RalA and SoS-mediated guanine nucleotide exchange of H-Ras and K-Ras by 50 μM compounds for 24 h incubation at 4° C., as illustrated in FIG. 3. Generally, the compounds inhibited wild-type RalB and RalA with similar potency, while showing weaker inhibition of RalB Tyr82Phe mutant and K-Ras exchange. Several compounds had substantially lower potency, such as Compounds 8, 17-19, and 20. As expected, Compound 17, which was synthesized to confirm that adduct formation was essential to inhibition, did not inhibit any of the proteins. For compounds 18 and 19, the removal of the sulfonamide group lead to loss of inhibition (FIG. 3). This sulfonamide moiety is important as it makes hydrogen bonds with the backbone nitrogens of Ala-70 and Gln-72 on RalB. The fluorosulfate derivative of Compound 1, namely Compound 20 also did not inhibit Ral exchange. This is attributed the shift in the position of the sulfur due to presence of an additional oxygen atom as well as the low intrinsic reactivity of the fluorosulfate reactive group. All other compounds showed significant inhibition of wild-type RalA and RalB, and were tested in a concentration-dependent manner to determine their IC₅₀ at 24 h as detailed in Table 1.

Compound 2 was a better inhibitor of wild-type RalB exchange than Compound 1. Compound 2 showed no inhibition of the RalB Tyr82Phe mutant. A crystal structure of Compound 2 in complex with RalA was determined at 1.30 Å resolution as indicated in Table 1; the electron density clearly identified the binding mode of the compound. The binding mode of Compound 2 reveals that the compound preserves the interaction from the sulfone to the backbone amides of Ala-70 and Gln-72. As the methoxy group is moved from the meta position to the ortho position, the clash with Phe-83 does not occur. In addition, a hydrogen-bond to the backbone amide of Glu-73 is established with the methoxy group at the ortho position. This additional hydrogen bond may explain the 2-fold improvement in IC₅₀ at 24 h for Compound 2 as indicated in Table 1.

Compound 3, which features a hydroxyl moiety at the ortho position, was a strong inhibitor of wild-type RalB, with an IC₅₀ of 17.0±7.7 μM at 24 h. However, this improvement in inhibition was accompanied by inhibition of the Tyr82Phe mutant, as well as K-Ras, which may indicate one or more off-target reactions.

Compound 4 has a similar IC₅₀ to Compound 1 of 41.7±8.5 μM. An X-ray crystal structure of Compound 4 in complex with RalA was determined at 1.50 Å. The compound was clearly present in the electron density. Compound 4 lacks the methoxy group present in Compound 1.

Compound 5 and Compound 6 possess halogen atoms at the meta position replacing the methoxy group of Compound 1. The chlorine derivative Compound 5 showed a 2-fold improvement in IC₅₀ compared to Compound 1 (IC₅₀=24.4±4.9 μM), while the fluorine derivative 6 had similar IC₅₀ to Compound 1 (51.5±5.2 μM). Crystal structures of RalA-5 and RalA-6 were determined at 1.49 Å and 1.63 Å, respectively as indicated in Table 1. The compounds were clearly present in the electron density. Structures of the bound compounds reveal that the hydrogen bonds with the sulfonamide are maintained and the clash with Phe-83 is alleviated. The chlorine fits better into the hydrophobic binding pocket better than the fluorine, perhaps making better van der Waals interactions. An additional nitrogen in Compound 7 negates the 2-fold improvement gained in Compound 5.

Substitutions at the para position has detrimental effects to the IC₅₀, probably due to clashes with Phe-83. This is seen in Compounds 8-10, 12 and 13. In the case of Compound 12, the change to a nitrogen atom may make the moiety unsuitable in the hydrophobic pocket. The modification of the methoxy group at the meta position in Compounds 14-16 resulted in weaker inhibition compared to Compound 1. The larger and more hydrophobic moieties likely result in increased clashes in the pocket.

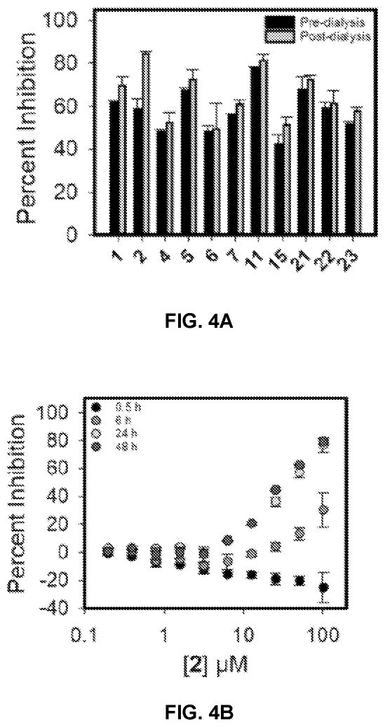

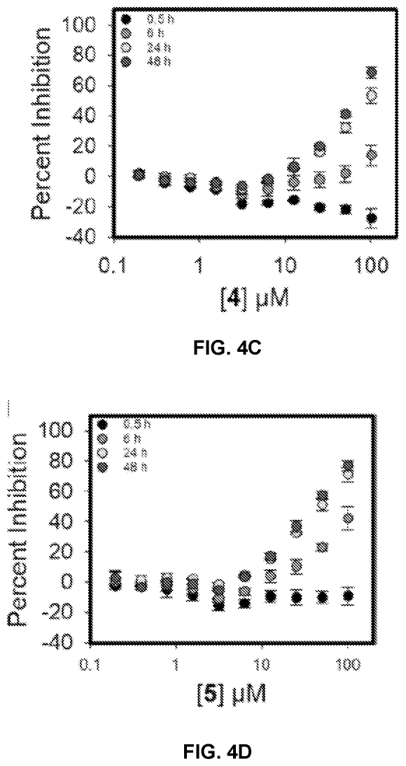

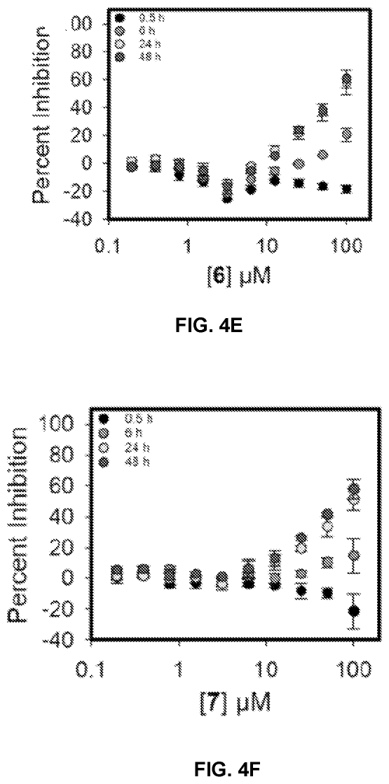

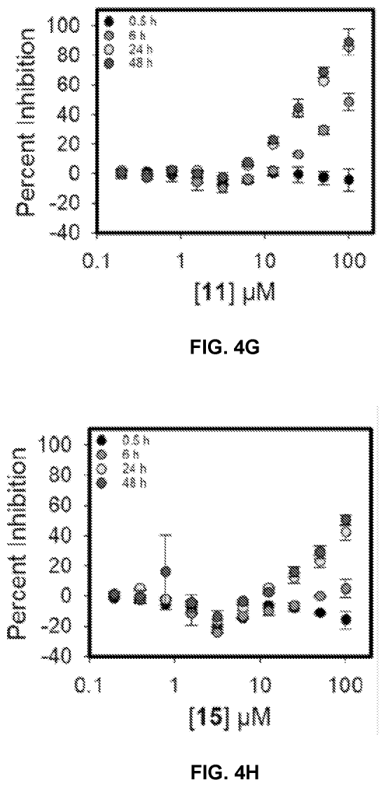

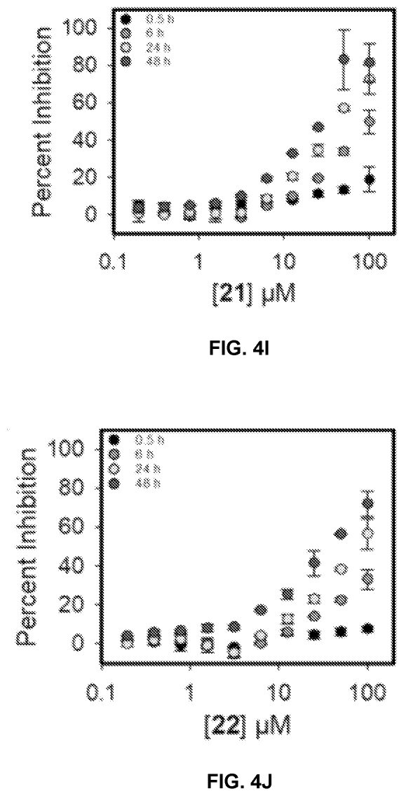

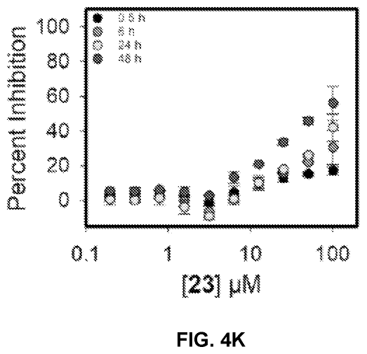

FIG. 4A illustrates percent inhibition of Rgl2-mediated guanine nucleotide exchange of RalB by 100 μM compounds after 24 h incubation at 4° C. followed by 24 h dialysis against assay buffer at 4° C. Supplementary FIGS. 4B-4K illustrate concentration-dependent percent inhibition of Rgl2-mediated guanine nucleotide exchange of RalB after 0.5, 6, 24 and 48 h incubation at 4° C. cmp 2 (FIG. 4B), cmp 4 (FIG. 4C), cmp 5 (FIG. 4D), cmp 6 (FIG. 4E) cmp 7 (FIG. 4F), cmp 11 (FIG. 4G) cmp 15 (FIG. 4H), cmp 21 (FIG. 4I) cmp 22 (FIG. 4J), and cmp 23 (FIG. 4K).

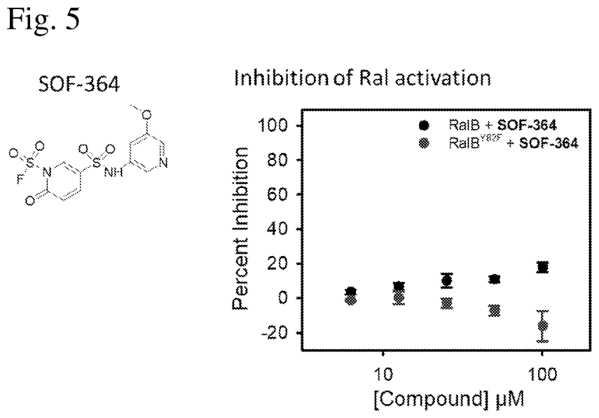

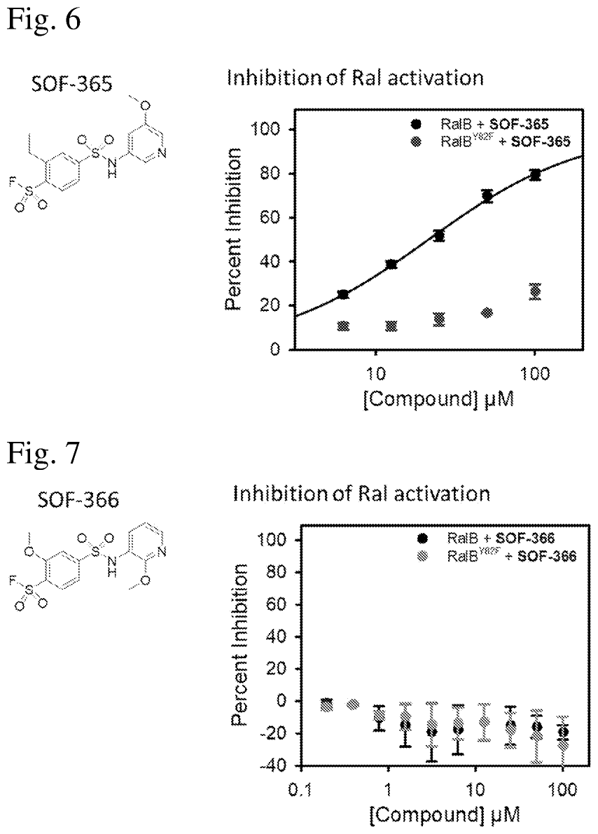

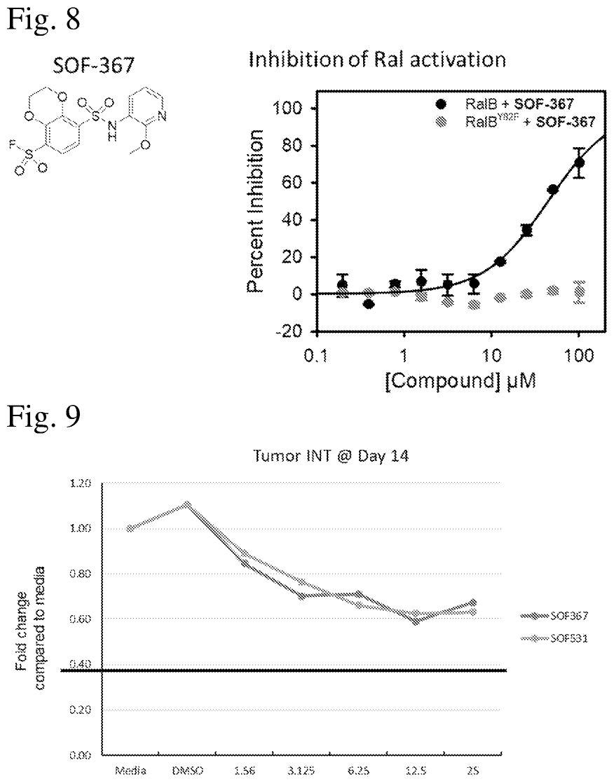

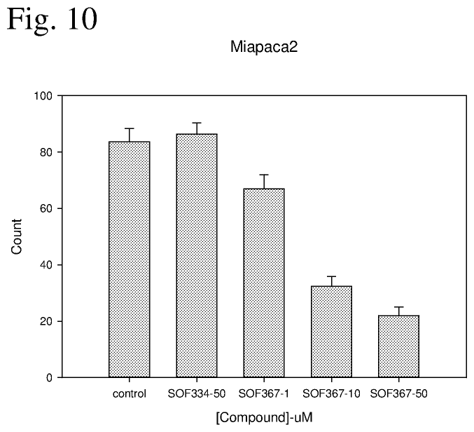

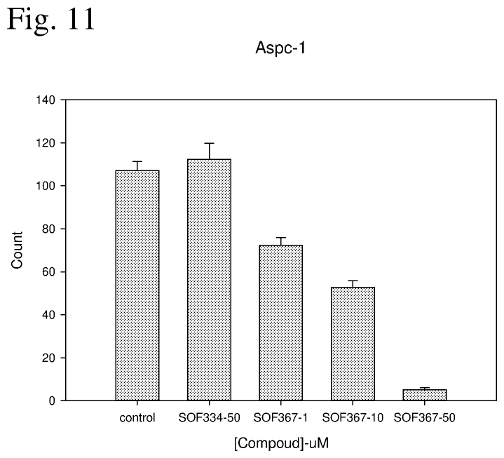

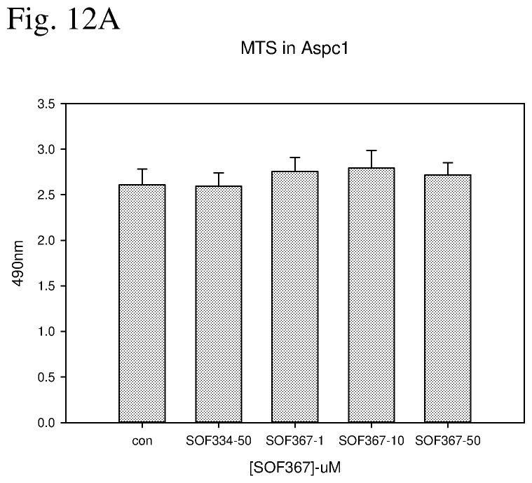

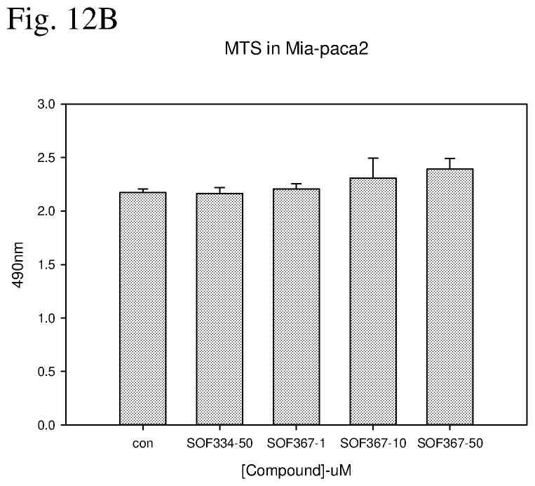

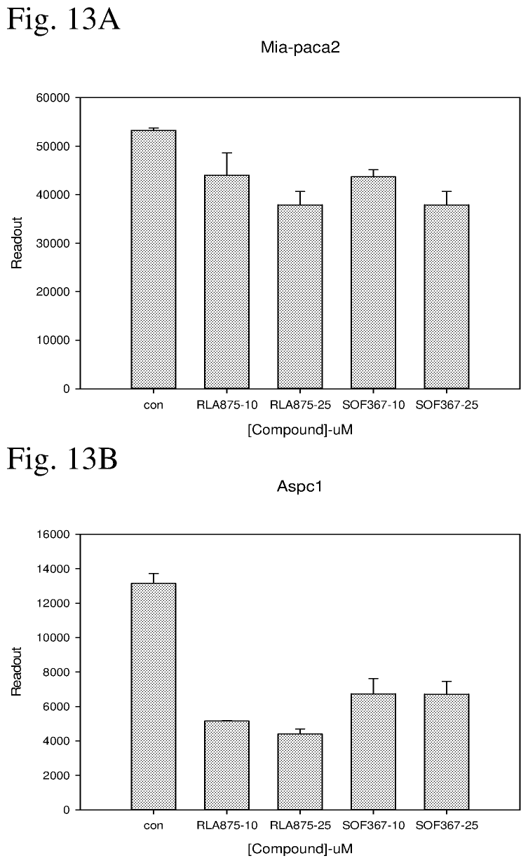

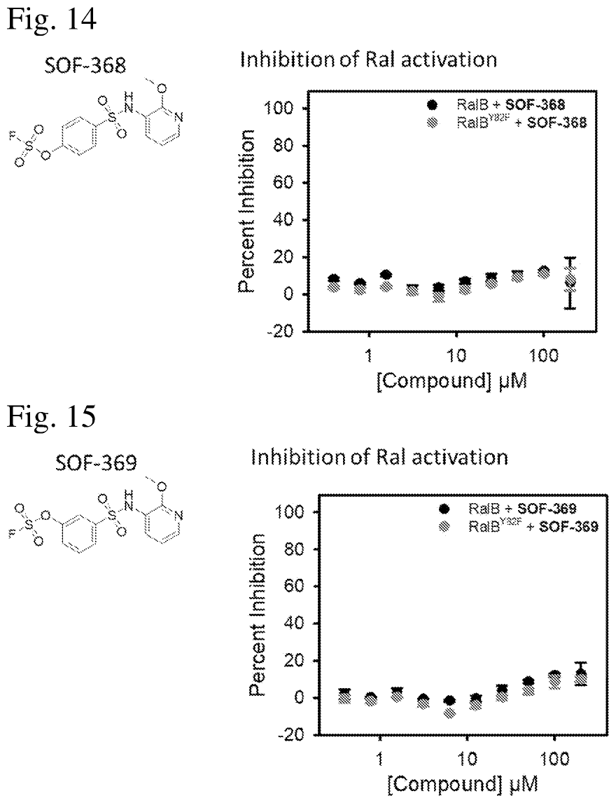

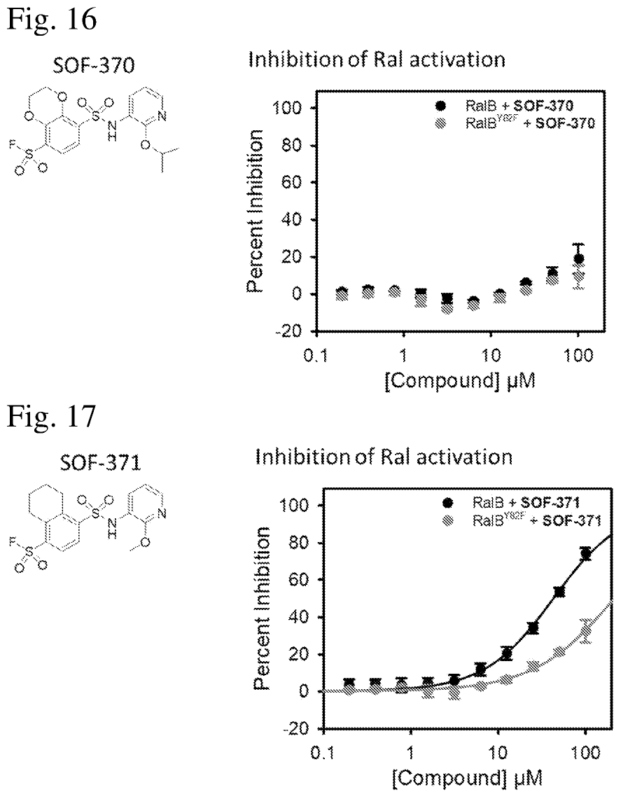

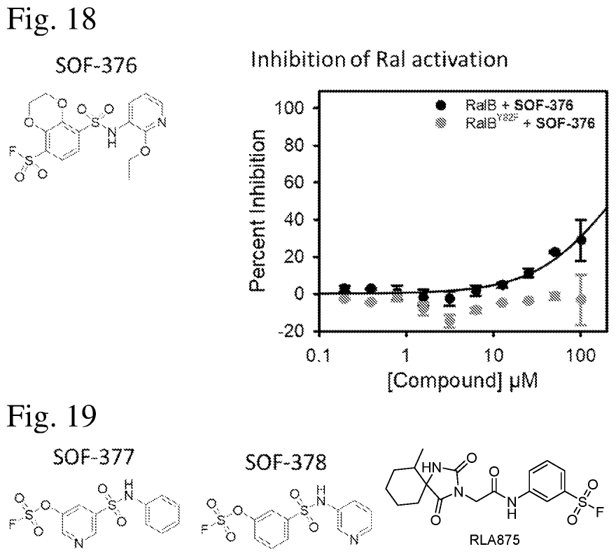

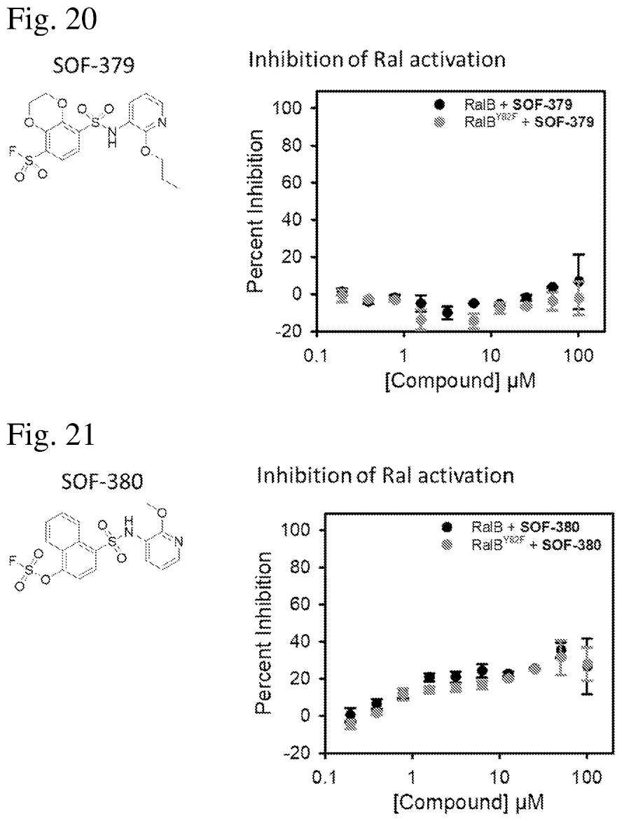

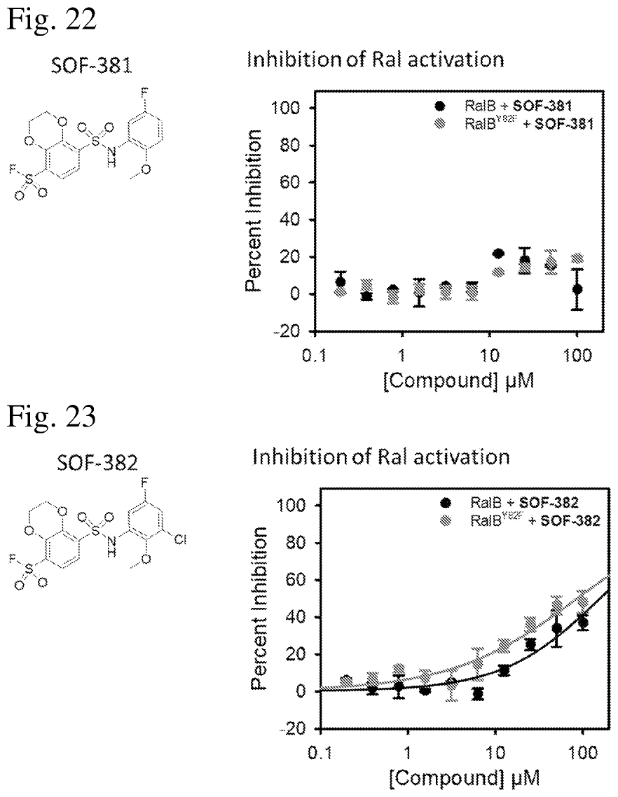

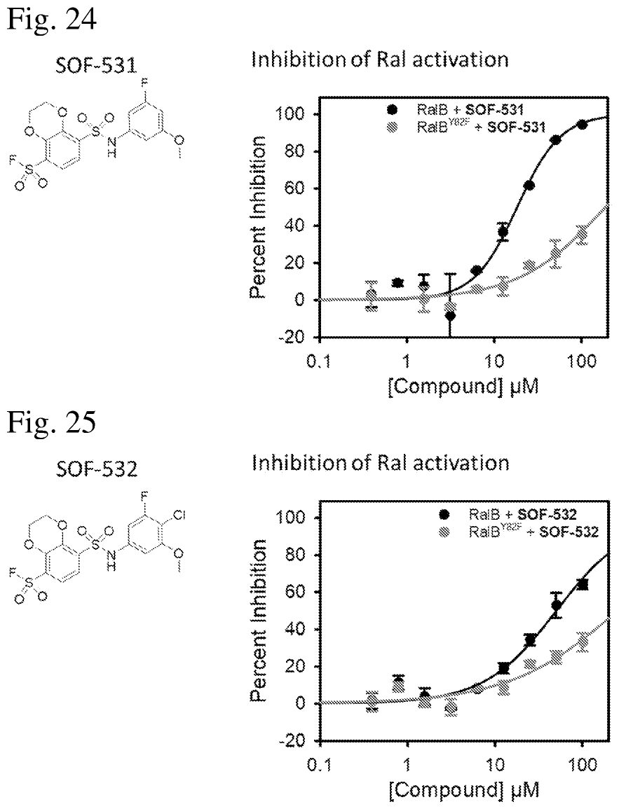

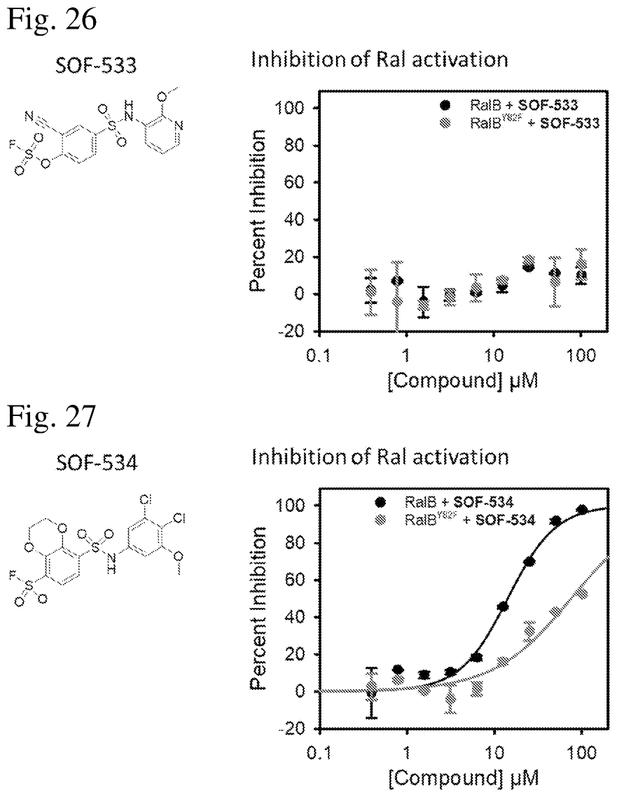

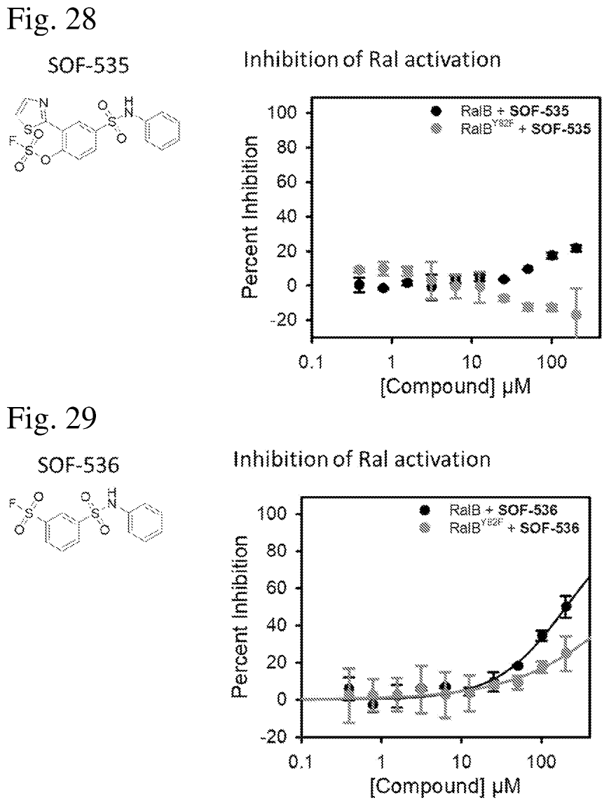

Additional RalB-inhibiting and K-Ras-inhibiting derivatives of Compound 1 are povided in FIGS. 5-29, along with accompanying additional biochemical and cell biological data for these derivatives.

### Additional Derivatives of Compound 1—RalB and K-Ras Inhibitors

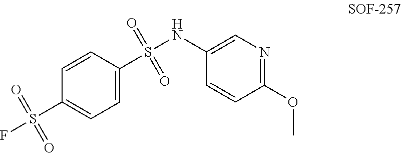 [SMILES: COc1ccc(NS(=O)(=O)c2ccc(S(=O)(=O)F)cc2)cn1]

LRMS (m/z) for C₁₂H₁₂FN₂O₅S₂⁺[M+H]⁺: calculated 347.0 found 347.0: ¹H NMR (400 MHz, CDCl₃): δ 8.13−8.11 (d, J=8.8 Hz, 2H), 7.97−7.95 (d, J=8.0 Hz, 2H), 7.76−7.75 (d, J=2.8 Hz, 2H), 7.46−7.43 (dd J=2.8 Hz, J=8.8 Hz, 1H), 6.74−6.72 (d, J=8.8 Hz, 2H), 6.54 (s, 1H), 3.90 (s, 3H).

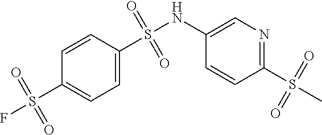 [SMILES: CS(=O)(=O)c1ccc(NS(=O)(=O)c2ccc(S(=O)(=O)F)cc2)cn1]

LRMS (m/z) for C₁₂H₁₂FN₂O₆S₃⁺[M+H]⁺: calculated 395.0 found 395.0: ¹H NMR (400 MHz, d₆-DMSO): δ 11.72 (br, 1H), 8.49 (d, J=2.0 Hz, 1H), 8.39−8.37 (d, J=8.4 Hz, 2H), 8.23−8.21 (d, J=8.4 Hz, 2H), 7.99−7.97 (d, J=8.8 Hz, 1H), 7.85−7.82 (dd, J=2.4 Hz, J=8.8 Hz 1H), 3.20 (s, 3H).

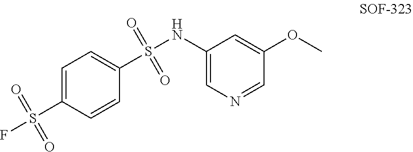 [SMILES: COc1cncc(NS(=O)(=O)c2ccc(S(=O)(=O)F)cc2)c1]

LRMS (m/z) for C₁₂H₁₂FN₂O₅S₂⁺[M+H]⁺: calculated 347.0 found 347.0: ¹H NMR (400 MHz, d₆-DMSO): δ 8.36−8.33 (d, J=8.4 Hz, 2H), 8.13−8.11 (d, J=8.0 Hz, 2H), 8.04−8.03 (d, J=2.4 Hz, 1H), 7.90 (d, J=1.6 Hz, 1H), 7.08 (d, J=2.4 Hz, 1H), 3.77 (s, 3H).

### Discussion of the Example Screening Method According to the Preceding Embodiment of this Disclosure

Ral (Ras-like) GTPases are directly activated by Ras GTPases through RalGEFs. Like Ras GTPases, binding sites on Ral GTPases are shallow and do not have the combination of size, hydrophobic and hydrophilic characteristics that are required to engage a drug at therapeutic doses.

One strategy to overcome the problem of a lack of druggable pockets is to develop covalent inhibitors. As discussed above, known efforts to develop small molecules that bind to either Ral or Ras has led to compounds with in vivo efficacy, but none of these compounds can engage Ral at therapeutic doses in a majority of Ral- or Ras-driven tumors. Analysis of Ral and Ras crystal structures in complex with their effector or GEFs revealed the presence of a tyrosine residue on Ral (Tyr-82) and Ras (Tyr-71) at the protein-protein interface. In addition, this tyrosine is located near a binding pocket that accommodates a tryptophan from BP1 and small-molecule fragments on K-Ras. It was hypothesized that small molecules that form a covalent bond at Tyr-82 could disrupt GEF-mediated activation of Ral GTPases.

To test this hypothesis, a library of aryl sulfonyl fluoride was screened to identify potential candidates that would form an adduct at Tyr-82. The methods of analyzing Ral and Ras crystal structures discussed herein, in complex with their effector or GEFs, revealed the presence of a tyrosine residue on Ral (Tyr-82) and Ras (Tyr-71) at the protein-protein interface. Unexpectedly and advantageously, this tyrosine is located near a binding pocket that accommodates a tryptophan on Ral and small-molecule fragments on K-Ras. As a result of the methods disclosed herein, small molecules have been identified that form a covalent bond at Tyr-82 could disrupt GEF-mediated activation of Ral GTPases. One goal of the example methods described herein was to determine if Tyr-82 was available for adduct formation and whether the binding sites near the tyrosine were accessible to small molecules for drug development targeting Ral and Ras. The discovery of Compound 1 and derivatives confirmed that Tyr-82 was amenable to adduct formation. Other potential candidates near Tyr-82 include Thr-58, which is present in both Ral and Ras.

Unexpectedly, these compounds uncovered a completely new and druggable pocket on Ral GTPases. This binding site is not present in any crystal structure of apo Ras or Ral GTPases or in complexes of these proteins with fragments and compounds. Instead, it was expected that these compounds would occupy the binding pocket of the tryptophan of RalPB1, where several fragments were identified to bind on the same pocket on K-Ras. The apo structure of RalA confirms that the switch II region of Ral is highly flexible, which is also the case among most members of the Ras and Rho GTPase families. Without Compound 1, it is unlikely that the pocket would have been identified. The chemical structure of the compound is also likely another reason for the discovery of the druggable pocket. Structure-activity studies based on synthesis of several derivatives and several high-resolution X-ray structures as disclosed herein reveal the importance of two key hydrogen bonding interactions by the sulfonamide oxygen atoms with backbone nitrogen atoms of Ral switch II residues Ala-70 and Gln-72.

Screening campaigns using small molecules lacking a reactive group have not identified the pocket possibly due to lack of diversity considering that compounds must have a combination of high affinity along with a binding mode to create key hydrogen bonding interactions. High affinity for non-covalent inhibitors will be essential, as evidenced by the fact that removing the reactive groups from Compound 1 led to inactive Compound 17. To achieve high affinity, non-covalent inhibitors may have to bind deeper into the pocket and extend into neighboring G12 binding site on K-Ras. A non-covalent inhibitor would also have to make key hydrogen bonding interactions with RalA backbone atoms similar to those of the sulfone oxygen atoms of Compound 1. The fact that our work uncovered the pocket while other efforts have not can be attributed to the use of covalent fragments. While fragments have generally lower affinity, their smaller size enable high complementarity in their binding. Fragments alone would not have sufficient binding affinity to trap the conformation of RalA and open the pocket, so the presence of a reactive group that formed an adduct with Tyr82 was another key reason that led to the discovery of the pocket. The formation of a covalent bond created an anchor to trap the covalent complex and compensated for the low affinity of fragments.

The discovery that Tyr-82 is accessible for covalent modification and the presence of a druggable pocket near the residue could have profound implications for the development of therapeutic agents targeting the Ras signaling pathway. The binding mode of Compound 1 and derivatives provides a new strategy to develop Ral GTPase antagonists that can lead to therapeutic agents targeting the Ras signaling pathway. First, the reactive group must exhibit greater stability in buffer, and ideally in plasma and microsome to be suitable for animal studies. Sulfonyl fluorides are prone to hydrolysis by water. One strategy to stabilize the reactive moiety is to introduce substituents on the aromatic ring ortho, meta, or para to the reactive group, which could reduce the nucleophilic character of the sulfone. Another strategy is to replace the sulfonyl fluoride with more stable moieties such as fluorosulfates, which are generally considered inert in aqueous solvent. Second, the binding affinity of the compound must be improved. This can be accomplished through a standard medicinal chemistry approach by adding substituents on the compound to enhance its binding affinity to RalB or by modifying its core structure. Finally, it is important that a covalent inhibitor possesses a favorable binding constant (K₁) and larger inactivation rate constant (k_{inact}). Generally, the second order rate constant k_{inact}/K₁ is considered to be the most important parameter to guide compound optimization. A covalent inhibitor with a cellular IC₅₀ under 1 μM and a 4-hour occupancy time-point could be expected to have a k_{inact}/Kᵢ of ˜100 M⁻¹sec⁻¹. Physiologically relevant values above 1000 M⁻¹sec⁻¹, with sufficiently optimized k_{inact} values, can be good candidates for in vivo experiments.

### Example 1

Protein Expression and Purification: RGL2 (50-514). BL-21 (DE3) E. coli cells containing RGL2 (50-514) in pGEX-6P-1 plasmid was grown in Terrific Broth at 37° C. until OD₆₀₀ reached 0.6. Protein expression was induced with 0.5 mM IPTG at 16° C. for 16-20 h. Cells were harvested by centrifugation and lysed by passing multiple times through a microfluidizer in a buffer containing 400 mM NaCl, 50 mM Tris pH 8.0, 10% glycerol and 8 mM β-mercaptoethanol. The sample was clarified by centrifugation at 35,000×g for 1 h at 4° C., prior to being loaded onto a 5 mL GSTrap HP column (GE, Boston, Mass., Catalog Number: 17528202). The column was then washed with buffer containing 200 mM NaCl, 20 mM Tris pH 8.0, 10% glycerol and 1 mM TCEP, prior to being eluted with the same buffer supplemented with 10 mM glutathione. The GST tag was cleaved by adding 1:100 w/w HRV-3C enzyme (ThermoFisher, Waltham, Mass., Catalog Number: 88946) to the eluted protein and dialyzing against 200 mM NaCl, 20 mM Tris pH 8.0, 1 mM TCEP for 48 h at 4° C. The sample was re-purified on the GSTrap HP column to remove the cleaved GST tag. Finally, the protein was further purified on a HiLoad 26/600 Superdex 200 pg SEC column (GE, Boston, Mass., Catalog Number: 28989336) with 200 mM NaCl, 20 mM Tris pH 8.0 and 1 mM TCEP as buffer.

HIS-RalA (1-178): The plasmid of pet21a(+)-RalA was transformed into competent E. coli BL21(DE3) strain. Bacteria culture was grown in LB medium at 37° C. to an OD₆₀₀ of approximately 0.6 and then induced with 0.5 mM IPTG at 32° C. for 5 h. Cells were collected by centrifugation and the pellet was lysed by micro-fluidizer in lysis buffer (phosphate buffer, pH 7.6, 2 mM MgCl₂). The His-RalA protein was purified at 4° C. using Ni-IMAC chromatography (HisTrap HP, GE Healthcare) and eluted with 500 mM imidazole in lysis buffer with a gradient method. After the fractions consisting of His-Ras were combined and concentrated, the protein was further purified using size exclusion chromatography (Superdex 200 pg, GE Healthcare) in 10 mM HEPES (pH 7.5), 10 mM NaCl, 5 mM MgCl₂, 1 mM DTE, 1 μM GDP. After purification protein HIS-RalA was concentrated to 25 mg/mL for crystallization.

HIS-RalB (12-185): The plasmid of pHIS-RalB was transformed into competent E. coli BL21(DE3) strain. Bacteria culture was grown in TB medium at 37° C. to an OD₆₀₀ of approximately 0.6 and then induced with 0.5 mM IPTG at 25° C. for 16 h. Cells were collected by centrifugation and the pellet was lysed by micro-fluidizer in lysis buffer (phosphate buffer, pH 7.6, 2 mM MgCl₂). The His-RalB protein was purified at 4° C. using Ni-IMAC chromatography (HisTrap HP, GE Healthcare) and eluted with 500 mM imidazole in lysis buffer with a gradient method. After the fractions consist of His-RalB was combined and concentrated, the protein was further purified using size exclusion chromatography (Superdex 200 pg, GE Healthcare) in 50 mM sodium phosphate buffer pH 7.6, 100 mM NaCl, 1 mM MgCl₂. The plasmids of pHIS-RalBS50A, pHIS-RalBT69A, pHIS-RalBY82F and pHIS-RalBS85A mutants were generated using site-directed mutagenesis. The corresponding proteins were expressed and purified following the same protocol as HIS-RalB.

HIS-Ras: The plasmid of preceiver-B01.2x-KRas was transformed into competent E. coli BL21(DE3) strain. Bacteria culture was grown in TB medium at 37° C. to an OD600 of approximately 0.6 and then induced with 0.5 mM IPTG at 25° C. for 16 h. Cells were collected by centrifugation and the pellet was lysed by micro-fluidizer in lysis buffer (phosphate buffer, pH 7.6, 2 mM MgCl₂). The His-Ras protein was purified at 4° C. using Ni-IMAC chromatography (HisTrap HP, GE Healthcare) and eluted with 500 mM imidazole in lysis buffer with a gradient method. After the fractions consist of His-RalB was combined and concentrated, the protein was further purified using size exclusion chromatography (Superdex 200 pg, GE Healthcare) in 20 mM Tris, pH 8.0, 100 mM NaCl, 2 mM MgCl₂. Then, the protein was concentrated and stored at −80° C. for further experiments.

HIS-SOS-cat (564-1049): The plasmid of ProEX HTb-SOScat was transformed into competent E. coli BL21(DE3) strain. Bacteria culture was grown in LB medium at 37° C. to an OD₆₀₀ of approximately 0.6 and then induced with 0.5 mM IPTG at 16° C. for 16 h. Cells were collected by centrifugation and the pellet was lysed by micro-fluidizer in lysis buffer (phosphate buffer, pH 7.6, 2 mM MgCl₂). The His-SOS-cat protein was purified at 4° C. using Ni-IMAC chromatography (HisTrap HP, GE Healthcare) and eluted with 500 mM imidazole in lysis buffer with a gradient method. After the fractions consist of His-RalB was combined and concentrated, the protein was further purified using size exclusion chromatography (Superdex 200 pg, GE Healthcare) in 20 mM Tris, pH 8.0, 100 mM NaCl, 2 mM MgCl₂. Then the protein was concentrated and stored at −80° C. for further experiments.

Exchange Assay: 10 μL of RalB (2 μM), RalA (2 μM), and Ras (0.8 μM) in an exchange assay buffer (100 mM NaCl, 10 mM MgCl₂, 20 mM Tris pH 8, 0.01% IGEPAL) were added to a 384-well, black, round bottom, low volume, polystyrene plate (Corning, Corning, N.Y., Catalog Number: 4515) and incubated with 2 μL of varying concentrations of compounds in the exchange assay buffer supplemented with 20% v/v DMSO for 24 h (unless otherwise specified) at 4° C. After the incubation, 5 μL of RGL2 (1 μM), SOS (0.4 μM) or buffer was added. Finally, 3 μL of Bodipy-FL-GDP (1.67 μM for RalA/B, 1.0 μM for Ras) was added and the fluorescence was read immediately on an Envision Multilabel Plate Reader (PerkinElmer, Waltham, Mass.) using a filter set with excitation and emission wavelengths of 485 and 535 nm, respectively, for 40 min at 30 sec intervals.

The fluorescence increase over time was fitted to an exponential function:

Fluorescence Intensity=Initial Fluorescence+Extent of binding (1−e^{-Rate×Time})

The rate constant of the exchange was calculated by fitting experimental values for Fluorescence Intensity and corresponding Time. The Initial Fluorescence was estimated from the initial reading of the fluorescence intensity from the experimental control sample without guanine exchange factor (GEF). The Extent of binding is the difference between the maximal fluorescence intensity of the DMSO control sample versus the initial fluorescence recorded for the No GEF control sample. Percent inhibition was calculated by comparing the rate constant of the compound inhibited sample versus the maximal DMSO control and the minimal control without GEF. Based on the plot of the percent inhibition versus compound concentration, a four-parameter logistic curve was fit to determine the IC50 values at 24 h incubation time.

$$Percent Inhibition = Minimum Inhibition + \frac{( Maximum Inhibition - Minimum Inhibition )}{1 + {( \frac{Compound Concentration}{IC 50} )}^{- HillSlope}}$$

Percent Inhibition and compound concentration are experimental values. Maximum inhibition is set at 100 percent as no plateau were achieved. Minimum inhibition value was data-dependent and were mostly found to be near 0 percent. Due to the fact that the compounds were covalent inhibitors and not classical reversible inhibitors, Hillslope value was not constrained.

Protein Mass Spectrometry: Compounds were incubated with 5 μM RGL2 or Tyr82Phe mutant in buffer (100 mM NaCl, 20 mM Tris pH 8.0, 10 mM MgCl₂, 2% DMSO) for 24 h (unless otherwise specified) at 4° C. After the incubation, the samples were centrifuged at 20,000×g for 10 min to remove precipitants prior to being injected into a Zorbax 300-SB C3 column (Agilent, Santa Clara, Calif.) on an Agilent 1200 liquid chromatography system (Agilent, Santa Clara, Calif.), using a gradient of Buffer A (H₂O with 0.1% Formic Acid) and Buffer B (acetonitrile with 0.1% Formic Acid), and the masses were detected on an Agilent 6520 Accurate Mass Q-TOF.

Compound Stability Assay. 200 μM Compound 1 was incubated in a buffer containing 20 mM Tris pH 8.0, 100 mM NaCl, 10 mM MgCl₂ at 4° C. for varying amounts time. After the incubation, the samples were centrifuged at 20,000×g for 10 min to remove precipitants prior to being injected into an Agilent EclipsePlus C18 RRHD column (Agilent, Santa Clara, Calif.) on an Agilent 1200 liquid chromatography system (Agilent, Santa Clara, Calif.), using a linear gradient from 100% Buffer A (H₂O with 0.1% Formic Acid) to 70% Buffer B (acetonitrile with 0.1% Formic Acid), and the masses were detected on an Agilent 6520 Accurate Mass Q-TOF.

Crystallization and Structure Determination: RalA.GDP crystals were grown using the hanging-drop vapor-diffusion method with a drop containing 20-25 mg/ml RalA⋅GDP and reservoir solution (0.2 M calcium acetate pH 5.5 and 18-22% PEG3350) at 20° C. The crystals appeared after two days. RalA-inhibitor complexes were obtained by soaking the crystals overnight in reservoir solution supplemented with 2-5 mM compounds. Crystals were harvested and cryo-protected in reservoir solutions supplemented with 20% glycerol or a mix of 10% glycerol and 10% ethylene glycol prior to being flash-cooled in liquid nitrogen. Diffraction data were collected at 100 K at the Beamline station 4.2.2 at the Advanced Light Source (Berkeley National Laboratory, CA) and were indexed, integrated, and scaled using XDS. The structure was solved by molecular replacement using PHASER and the simian RalA model (PDB ID: 1U8Z). The Autobuild function was used to generate a first model that was improved by iterative cycles of manual building in Coot and refinement using PHENIX. MolProbity software was used to assess the geometric quality of the models and PyMOL (version 2.3.1) was used to generate molecular images. Data collection and refinement statistics are indicated in Table 1.

Single crystals were used to obtain a complete data set for each RalA-compound complex. In the case of apo RalA, data from three different crystals were collected and analyzed individually (crystal 1 at resolution 1.55 Å, crystal 2 at 1.54 Å [PBD ID: 6P0O] and crystal 3 at 1.31 Å [PDB ID: 6P0J]). For simplicity, two models (PDB ID: 6P0O “open” and PDB ID: 6P0J “closed” conformation) representing two distinct conformations were deposited.

Computational Analysis of Binding Sites: Binding sites were identified and scored as previously described. Identification of druggable binding sites on the crystal structures was carried out using the Schrodinger Software Suite (Small-Molecule Drug Discovery Suite 2019-1, Schrödinger, LLC, New York, N.Y., 2019). Structures were retrieved from the Protein Data Bank (PDB) and prepared using the Protein Preparation Wizard workflow in Maestro. Missing side chains and loops were added with the Prime module. Protein and ligand structures were protonated at pH 7.0 using PROPKA and Epik, respectively. The SiteMap module was used to evaluate the binding site of the compound on the prepared structure. The covalent bond between Compound 1 and Tyr-82 in the RalA-1 structure was removed. The region around the compound plus an additional 6 Å buffer was evaluated for potential binding sites. All other parameters were left to their default setting.

Binding sites are identified in SiteMap by overlaying a three-dimensional grid around the region. Each point of the grid (site point) is evaluated using van der Waals energies. Points are linked together to form the putative binding site. Each site is evaluated based on its ability to bind a ligand (SiteScore) and its druggability (DrugScore). Both SiteScore and DrugScore use the weighted sums of three parameters, namely the (i) number of site points in the binding site; (ii) enclosure score that is a measure of how open the binding site is to solvents; and (iii) hydrophilic character of the binding site (hydrophilic score). Unlike DrugScore, SiteScore limits the impact of hydrophilicity in charged and highly polar sites. A binding site with SiteScore and DrugScore of 0.8 is considered to be able to fit a small molecule ligand. SiteScore and DrugScore values closer to 0.8 are considered ‘difficult’ to drug, while binding sites with SiteScore and DrugScore closer to 1.1 are classified as highly ‘druggable’.

Based upon the foregoing disclosure, it should now be apparent that the small molecules for covalent inhibition of Ral, screening methods for identifying such compounds, and treatment methods for treating patients in need of such compounds, will carry out the objects set forth hereinabove. Namely, these small molecules for covalent inhibition of Ral are capable of serving as the basis for developing therapeutic agents to treat a range of tumors that are Ral- or Ras-driven. This includes pancreatic ductal adenocarcinoma, non-small cell lung cancer, neurofibromatosis, and colon cancer, but is not limited to these. It is, therefore, to be understood that any variations evident fall within the scope of the present disclosure and thus, the selection of specific component elements can be determined without departing from the spirit of the invention herein disclosed and described.

Invasion Assay: Invasion assays were performed using BD Biocoat Matrigel invasion chambers (BD Biosciences, San Jose, Calif.). The undersurface of the inserts was coated with 30 ng μl-1 of fibronectin at 4° C. overnight. The inserts were equilibrated with 0.5 mL of serum-free medium in the upper and lower chamber separately for 2 h at 37° C. After 4 h of serum starvation, cells were harvested and 5×10⁴ cells in 500 μL medium containing 0.1% FBS and the compounds at the indicated concentrations or 1% DMSO control were plated onto the upper chamber. As a control of cell viability, 104 cells at the above conditions were plated in 100 μl in each well of 96-well plates. 500 μL of 10% FBS medium containing the same amount of compounds or DMSO control was added to the lower chamber. After a 16 h incubation at 37° C. in 5% CO2, non-invaded cells were removed from the upper chamber with a cotton swab, and the invaded cells were fixed in methanol for 30 min at room temperature and stained with Hematoxylin Stain Harris Modified Method (Fisher Scientific, Waltham, Mass.) for 1 h at room temperature. We washed the filters with water 3 times. Filters were air-dried, and the number of invaded cells was counted in ten separate 200×fields; meanwhile, 20 μl 5 mg/ml MTT (Sigma-Aldrich, St. Louis, Mo.) were added to each well, cells were incubated at 37° C. in 5% CO₂ for 2 h, viable cells were quantified at absorbance of 570 nm and 630 nm (reference background) as previously described.

1. A method of inhibiting a Ral GTPase said method comprising the step of contacting said Ral GTPase with a compound having the structure of 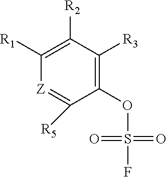
wherein
R₁ is H, 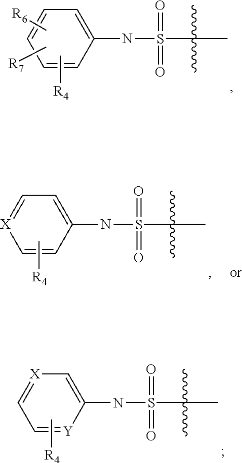
R₂ is H, C₁-C₄ alkyl, or CF₃, 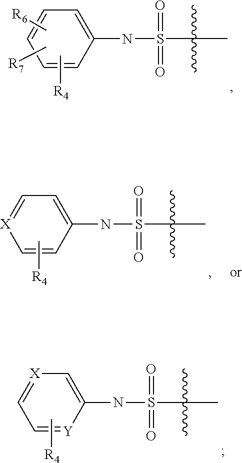
with the proviso that only one of R₁ or R₂ is 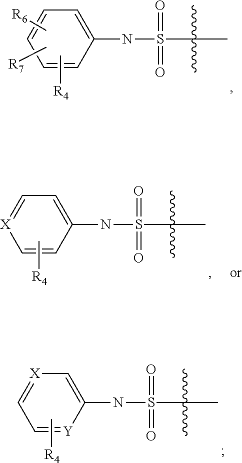
R₃ is H, —OCH₃, —OCH₂CH₃, C₁-C₄ alkyl, CH₂(morpholino) or R₂ and R₃ together with the atom to which they are attached form a 5 to 6 membered cyclic, 5 to 6 membered heterocyclic, or 5 to 6 membered aryl ring, optionally forming a 1,4 dioxane, cyclohexane, morpholino, or piperazinyl ring;
R₄ is C₁-C₄ alkyl, —OCH₃, —OCH₂CH₃, —OCH(CH₃)₂, —(SO₂)CH₃, —OH, or halo;
R₅ is H or ═O;
R₆ and R₇ are independently halo;
X, Y and Z are independently C or N, optionally wherein Z is N and X and Y are each C, optionally wherein X is N and Y and Z are each C and optionally wherein X, Y and Z are each C.

2. The method of claim 1 wherein
i) R₅ is H; and Z is C or
ii) R₅ is H; and Z is N.

3. (canceled)

4. The method of claim 1 wherein R₁ and R₂ are independently H or 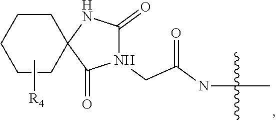
with the proviso that one of R₁ or R₂ is H.

5. The method of claim 1 wherein
i) R₁ is 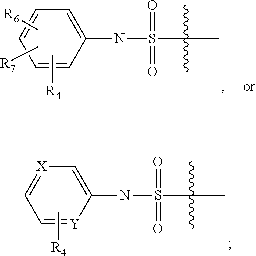
R₃ is H, C₁-C₄ alkyl, —OCH₃, —OCH₂CH₃, —OCH(CH₃)₂;
R₂ is H, or R₂ and R₃ together with the atom to which they are attached form a 1,4 dioxane or hexacyclic ring;
R₄ is C₁-C₄ alkyl, —OCH₃, —OCH₂CH₃, —OCH(CH₃)₂, or —(SO₂)CH₃;
R₆ and R₇ are independently halo or H, optionally wherein said halo is Cl or F; and
X and Y are independently C or N, optionally wherein X is N and Y is C, or
ii) R₁ is 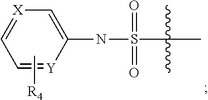
R₂ is H;
R₃ is H, C₁-C₄ alkyl, —OCH₃, —OCH₂CH₃, or —OCH(CH₃)₂;
R₄ is —OCH₃, —OCH₂CH₃, —OCH(CH₃)₂, —OH, or halo;
X and Y are independently C or N, optionally wherein X is N and Y is C or
iii) R₁ is 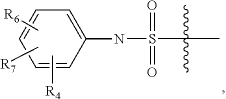
R₂ and R₃ together with the atom to which they are attached form a 1,4 dioxane or hexacyclic ring;
R₄ is —OCH₃, —OCH₂CH₃, —OCH(CH₃)₂, or —OH;
R₆ and R₇ are independently H or halo, optionally wherein said halo is F or Cl, optionally wherein R₆ and R₇ are each H, and
X and Y are independently C or N, optionally wherein X is N and Y is C, optionally wherein R₆ is H, X is N and Y is C, or
iv) R₁ is 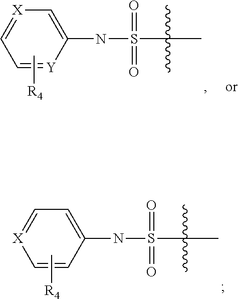
R₂ is H, C₁-C₄ alkyl, or CF₃;
R₃ is H or R₂ and R₃ together with the atom to which they are attached form a 6 membered heterocyclic, aryl ring, optionally a 1,4 dioxane, cyclohexane, morpholino, or piperazinyl ring;
R₄ is H, C₁-C₄ alkyl, —OCH₃, —OCH₂CH₃, —OCH(CH₃)₂, —(SO₂)CH₃, —OH, or halo;
X and Y are independently C or N, with the proviso that X and Y are not both N.

6-9. (canceled)

10. The method of claim 1 wherein said compound has the structure of: 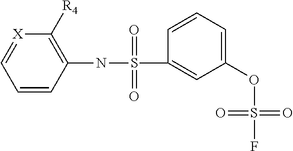
wherein
X is N or C; and
R₄ is H, —OCH₃, —OCH₂CH₃, —OCH(CH₃)₂, —OH, or halo.

11. The method of claim 1 comprising contacting a Ral GTPase with a compound selected from the group consisting of

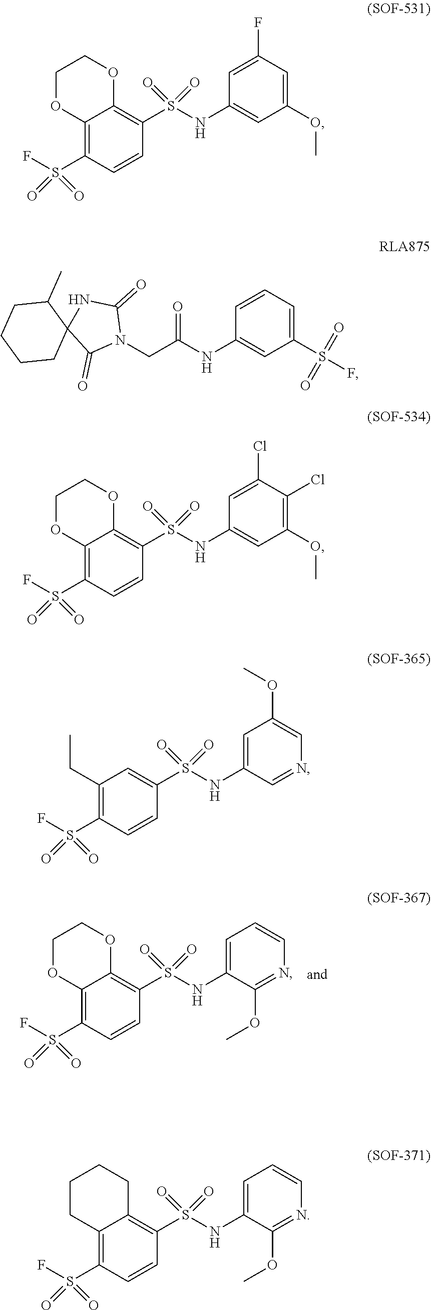 [SMILES: COc1cc(F)cc(NS(=O)(=O)c2ccc(S(=O)(=O)F)c3c2OCCO3)c1 ; CC1CCCCC12NC(=O)N(CC(=O)Nc1cccc(S(=O)(=O)F)c1)C2=O ; COc1cc(NS(=O)(=O)c2ccc(S(=O)(=O)F)c3c2OCCO3)cc(Cl)c1Cl ; CCc1cc(S(=O)(=O)Nc2cncc(OC)c2)ccc1S(=O)(=O)F ; COc1ncccc1NS(=O)(=O)c1ccc(S(=O)(=O)F)c2c1OCCO2 ; COc1ncccc1NS(=O)(=O)c1ccc(S(=O)(=O)F)c2c1CCCC2]

12. The method of claim 1 wherein said compound has the structure of: 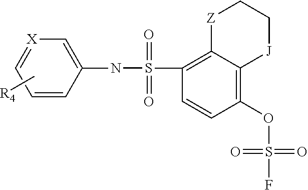
wherein
X is N or C;
Z and J are independently O, N or C; and
R₄ is —OCH₃, —OCH₂CH₃, —OCH(CH₃)₂, —OH, or halo.

13. The method of claim 1 wherein said compound has the structure of: 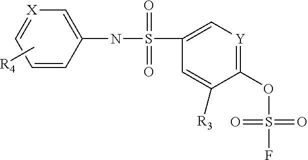
wherein
X and Y are independently N or C;
R₃ is H, —OCH₃, —OCH₂CH₃, C₁-C₄ alkyl; and
R₄ is C₁-C₄ alkyl, —OCH₃, —OCH₂CH₃, —OCH(CH₃)₂, —(SO₂)CH₃, —OH, or halo.

14. The method of claim 13 wherein said compound has the structure 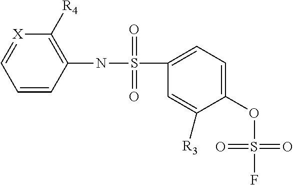
wherein
X is N or C;
R₃ is H, or —OCH₃; and
R₄ is —OCH₃, —OCH₂CH₃, —OCH(CH₃)₂, —OH, or halo.

15. The method of claim 1 wherein said compound has the structure of: 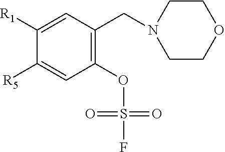
wherein R₁ is H, —OCH₃, —OCH₂CH₃, or —COCH₃; and
R₅ is H or R₁ and R₅ together with the atoms to which they are attached form a 5 to 6 membered cycloalkyl, 5 to 6 membered heterocyclic, or 5 to 6 membered aryl ring, optionally forming a 1,4 dioxane, cyclohexane, benzene, or piperazinyl ring.

16. A pharmaceutical composition comprising a compound of a compound having the structure of 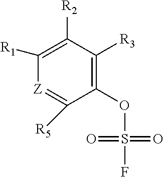
wherein
R₁ is H, 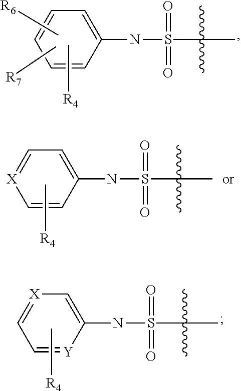
R₂ is H, C₁-C₄ alkyl, or CF₃, 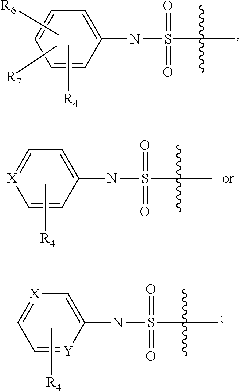
with the proviso that only one of R₁ or R₂ is 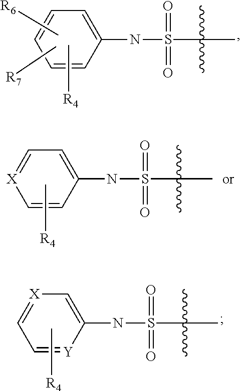
R₃ is H, —OCH₃, —OCH₂CH₃, C₁-C₄ alkyl, CH₂(morpholino) or R₂ and R₃ together with the atom to which they are attached form a 5 to 6 membered cyclic, 5 to 6 membered heterocyclic, or 5 to 6 membered aryl ring, optionally forming a 1,4 dioxane, cyclohexane, morpholino, or piperazinyl ring;
R₄ is C₁-C₄ alkyl, —OCH₃, —OCH₂CH₃, —OCH(CH₃)₂, —(SO₂)CH₃, —OH, or halo;
R₅ is H or ═O;
R₆ and R₇ are independently halo;
X, Y and Z are independently C or N, optionally wherein Z is N and X and Y are each C, optionally wherein X is N and Y and Z are each C and optionally wherein X, Y and Z are each C.

17. The pharmaceutical composition of claim 16 wherein
i) R₅ is H; and Z is C or
ii) R₅ is H; and Z is N.

18. (canceled)

19. The pharmaceutical composition of claim 16 wherein
R₁ and R₂ are independently H or 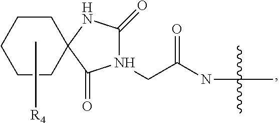
with the proviso that one of R₁ or R₂ is H.

20. The pharmaceutical composition of claim 16 wherein
i) R₁ is 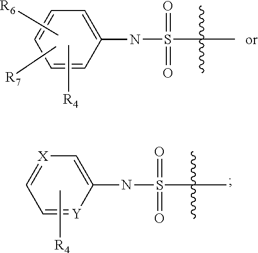
R₃ is H, C₁-C₄ alkyl, —OCH₃, —OCH₂CH₃, —OCH(CH₃)₂;
R₂ is H, or R₂ and R₃ together with the atom to which they are attached form a 1,4 dioxane or hexacyclic ring;
R₄ is C₁-C₄ alkyl, —OCH₃, —OCH₂CH₃, —OCH(CH₃)₂, or —(SO₂)C₃;
R₆ and R₇ are independently halo or H, optionally wherein said halo is Cl or F; and
X and Y are independently C or N, optionally wherein X is N and Y is C, or
ii) R₁ is 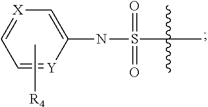
R₂ is H;
R₃ is H, C₁-C₄ alkyl, —OCH₃, —OCH₂CH₃, or —OCH(CH₃)₂;
R₄ is —OCH₃, —OCH₂CH₃, —OCH(CH₃)₂, —OH, or halo;
X and Y are independently C or N, optionally wherein X is N and Y is C or
iii) R₁ is 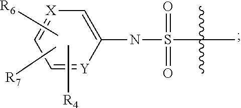
R₂ and R₃ together with the atom to which they are attached form a 1,4 dioxane or hexacyclic ring;
R₄ is —OCH₃, —OCH₂CH₃, —OCH(CH₃)₂, or —OH;
R₆ and R₇ are independently H or halo, optionally wherein said halo is F or Cl, optionally wherein R₆ and R₇ are each H, and
X and Y are independently C or N, optionally wherein X is N and Y is C, optionally wherein R₆ is H, X is N and Y is C or
iv) R₁ is 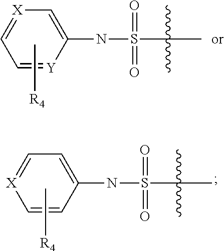
R₂ is H, C₁-C₄ alkyl, or CF₃;
R₃ is H or R₂ and R₃ together with the atom to which they are attached form a 6 membered heterocyclic, aryl ring, optionally a 1,4 dioxane, cyclohexane, morpholino, or piperazinyl ring;
R₄ is H, C₁-C₄ alkyl, —OCH₃, —OCH₂CH₃, —OCH(CH₃)₂, —(SO₂)CH₃, —OH, or halo;
X and Y are independently C or N, with the proviso that X and Y are not both N.

21-24. (canceled)

25. The pharmaceutical composition of claim 16 wherein said compound is selected from the group consisting of

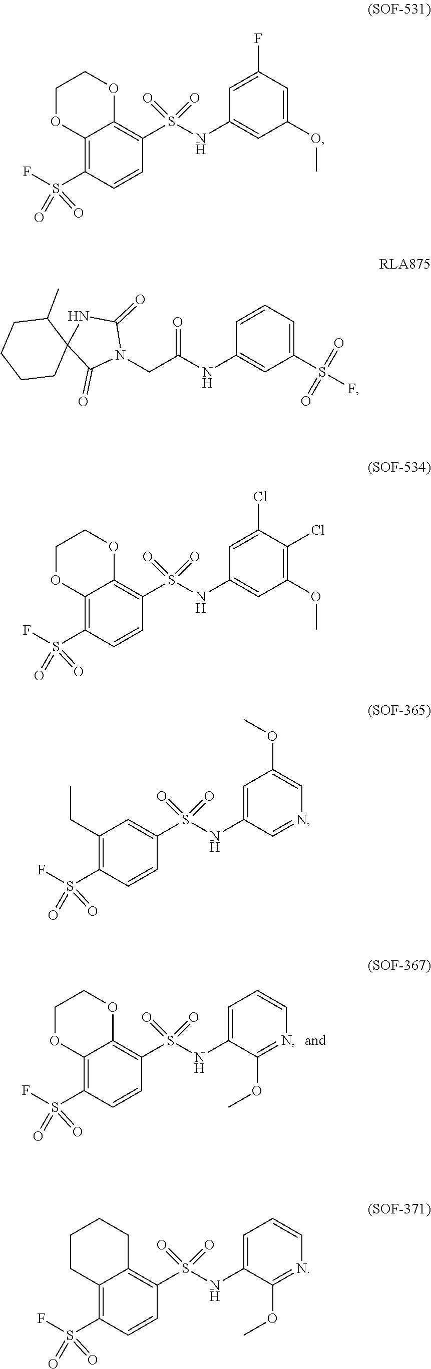 [SMILES: COc1cc(F)cc(NS(=O)(=O)c2ccc(S(=O)(=O)F)c3c2OCCO3)c1 ; CC1CCCCC12NC(=O)N(CC(=O)Nc1cccc(S(=O)(=O)F)c1)C2=O ; COc1cc(NS(=O)(=O)c2ccc(S(=O)(=O)F)c3c2OCCO3)cc(Cl)c1Cl ; CCc1cc(S(=O)(=O)Nc2cncc(OC)c2)ccc1S(=O)(=O)F ; COc1ncccc1NS(=O)(=O)c1ccc(S(=O)(=O)F)c2c1OCCO2 ; COc1ncccc1NS(=O)(=O)c1ccc(S(=O)(=O)F)c2c1CCCC2]

26. The pharmaceutical composition of claim 16 wherein said compound has the structure of: 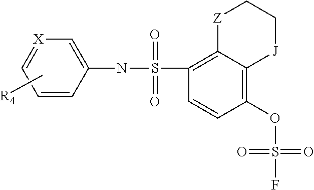
wherein
X is N or C;
Z and J are independently O, N or C; and
R₄ is —OCH₃, —OCH₂CH₃, —OCH(CH₃)₂, —OH, or halo.

27. The pharmaceutical composition of claim 16 wherein said compound has the structure of: 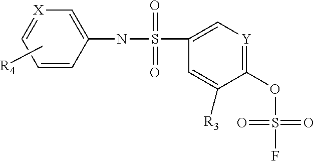
wherein
X and Y are independently N or C;
R₃ is H, —OCH₃, —OCH₂CH₃, C₁-C₄ alkyl; and
R₄ is C₁-C₄ alkyl, —OCH₃, —OCH₂CH₃, —OCH(CH₃)₂, —(SO₂)CH₃, —OH, or halo.

28. The pharmaceutical composition of claim 16 wherein said compound has the structure of: 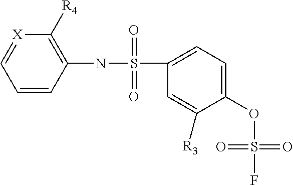
wherein
X is N or C;
R₃ is H, or —OCH3; and
R₄ is —OCH₃, —OCH₂CH₃, —OCH(CH₃)₂, —OH, or halo.

29. The composition of claim 16 wherein said compound has the structure of: 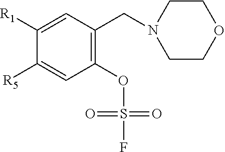
wherein R₁ is H, —OCH3, —OCH2CH3, or —COCH3; and
R₅ is H or R₁ and R₅ together with the atoms to which they are attached form a 5 to 6 membered cycloalkyl, 5 to 6 membered heterocyclic, or 5 to 6 membered aryl ring, optionally forming a 1,4 dioxane, cyclohexane, benzene, or piperazinyl ring.

30. The composition of claim 16 further comprising a chemotherapeutic agent.

31. A method of treating cancer said method comprising the step of administering a pharmaceutical compound of claim 16.

32-39. (canceled)
